(19) United States
(12) Patent Application Publication
(10) Pub. No.: US 2014/0342362 A1
(43) Pub. Date: Nov. 20, 2014

(54) COMPOSITIONS, KITS, AND RELATED METHODS FOR DETECTING AND/OR MONITORING SHIGA TOXIN PRODUCING ESCHERICHIA COLI

(71) Applicant: Roka Bioscience, Inc., Warren, NJ (US)

(72) Inventors: Michael McClellan Becker, San Diego, CA (US); Bettina Groschel, San Diego, CA (US)

(21) Appl. No.: 14/369,888

(22) PCT Filed: Dec. 31, 2012
(86) PCT No.: PCT/US12/72329

§ 371 Date: Jun. 30, 2014

Related U.S. Application Data
(60) Provisional application No. 61/582,153, filed on Dec. 30, 2011.
(60) Provisional application No. 61/646,180, filed on May 11, 2012.

(51) Int. Cl. C12Q 1/68

(52) U.S. Cl. CPC C12Q 1/689; C12Q 2600/158
USPC 435/6.11; 536/23.1

(57) ABSTRACT

ECF, such as the ecf operon/gene cluster (e.g., ECF2-1- and ECF2-2 described herein) may be used to detect virulent STECs including virulent non-0157:H7 STEC and virulent non-0157:H7 EHEC. Use of this nucleic acid target, in combination with other targets, such as Z5866, rfb₀₁₅₇, wzx₀₁₅₇, wzy₀₁₅₇, Z0344, Z0372, SIL⁰¹⁵⁷, and katP junction provides a robust, sensitive assay for distinguishing 0157:H7 from virulent non-0157:H7 STEC. Compositions, kits and methods used for the detection of E. coli STEC are further disclosed. Certain E. coli sequences were surprisingly efficacious for the detection of 0157:H7 and virulent non-0157 STECs, such as the big six: 026, 045, 0103, 0111, 0121, and 0145.

### SEQUENCE LISTING

The instant application contains a Sequence Listing which has been submitted in ASCII format via EFS-Web and is hereby incorporated by reference in its entirety. Said ASCII copy, created on Dec. 28, 2012, is named 5667135W.txt and is 38,833 bytes in size.

### BACKGROUND OF THE INVENTION

This invention relates to detection or monitoring or both of Shiga toxin producing E. coli (“STEC”), for example, by using in vitro nucleic acid amplification and detection of amplified sequences.

There are more than 200 Shiga toxin (stx)-producing Escherichia coli (“STEC”) serotypes, but many have not been implicated in causing illness. STEC may cause devastating illnesses, particularly in children, of varying severity, from diarrhea (often bloody), hemorrhagic colitis, and abdominal cramps to kidney disorders. Outbreaks of illnesses caused by STEC have been epidemiologically related to contact with animals and consumption of meat and fresh produce. Shiga toxin will bind to tissues in the kidneys and cause hemolytic uremic syndrome (“HUS”), leading to kidney failure and death. STEC also may cause asymptomatic infections and extraintestinal infections. Enterohemorrhagic E. coli (“EHEC”) is a subset of STEC and includes well recognized human pathogens. EHEC infections, like STEC infections, result in hemorrhagic colitis, which may progress into life-threatening HUS. E. coli O₁₅₇:H7 is the most notorious STEC/EHEC strain most often associated with the most severe forms of disease. O₁₅₇:H7 is a known food-borne pathogen increasingly causing illness worldwide.

Numerous non-0157 STEC isolates have also been linked to illnesses and outbreaks of disease. Six O groups have been described by the U.S. Center for Disease Control (“CDC”) to be the cause of the majority of non-O157 STEC disease. These serotypes have been identified as O26, O45, O103, O111, O121, and O145, and are commonly referred to as the “big six” non-O157 STEC. It is estimated that non-O157 STEC may cause diarrhea at frequencies similar to other enteric bacterial pathogens, such as Salmonella and Shigella. Non-O157 STEC also causes infections resulting in HUS.

The morbidity and mortality associated with worldwide outbreaks of STEC disease have highlighted the threat these organisms pose to public health. For this reason, there is a demand for compositions and diagnostic methods for detection of STEC in environmental and biological samples and, in particular, in foods such as meat and dairy products. Accordingly, there remains a need in the art for a rapid and robust detection system that can specifically and selectively identify virulent E. coli STEC in a sample of interest including virulent non-O157:H7 STECs O26, O45, O103, O111, O121, and O145.

### SUMMARY OF THE INVENTION

In general, as is described herein, the invention relates to the use of ECF such as the ecf operon/gene cluster (e.g., ECF2-1 and ECF2-2 described herein) to detect virulent STECs including virulent non-O157:H7 STEC and virulent non-O157:H7 EHEC. Use of this nucleic acid target, in combination, with other targets such as Z5866, rfb_{O157}, wzx_{O157}, wzy_{O57}, Z0344, Z0372, SIL_{O157}, and katP junction provides a robust, sensitive assay for distinguishing O157:H7 from virulent non-O157:H7 STEC.

The invention accordingly relates to compositions, kits, and methods used for the detection of E. coli STEC. The invention is based at least in part on the discovery that certain E. coli sequences are surprisingly efficacious for the detection of O157:H7 and virulent non-O157 STECs such as the big six: O26, O45, O103, O111, O121, and O145. In certain aspects and embodiments, particular regions of O157:H7 STEC have been identified as useful targets for nucleic acid amplification and, which when used in combination, provide improvements in relation to specificity, sensitivity, or speed of detection as well as other advantages.

By “virulent non-O157:H7 STEC” is meant any E. coli bacterium containing an Ed gene cluster other than O157:H7. Exemplary virulent non-O157:H7 STEC include E. coli such as O26, O45, O103, O111, O121, and O145. Other exemplary non-O157:H7 STEC are those containing stx1 or stx2 in combination with eae and ehxA (hlyA).

In one aspect, the invention features a first method for assigning whether a sample includes Shiga-toxin producing E. coli (STEC), the method includes the steps of: (a) providing nucleic acids from a sample; (b) detecting an O157-specific fragment and an ECF-specific fragment; (c) assigning to the sample one of the following outcomes: 1) if the O157-specific fragment and the ECF-specific fragment are absent then the sample is negative for virulent O157 STEC and a virulent non-O157:H7 STEC; 2) if the O157-specific fragment is present and the ECF-specific fragment is absent then the sample is negative for a virulent non-O157:H7 STEC; 3) if the O157-specific fragment and ECF-specific fragment are present then the sample includes virulent O157 STEC; or 4) if the O157-specific fragment is absent and the ECF-specific fragment is present then the sample includes a virulent non-O157:H7 STEC. This method typically includes an O157-specific fragment which is rfb, wzx, or wzy as is disclosed herein. Exemplary virulent O157 STEC include O157:H7, O157:NM, O157:H-, O157:H8, or O157:H21. And exemplary virulent, non-O157:H7 STEC includes O26, O45, O103, O111, O121, or O145. The method also involves detection of at least two O157-specific fragments (e.g., rfb and wzk, rfb and wzy, and wzk and wzy, or rfb, wzk, and wzy). Other exemplary O157-specific fragments include katP junction and Z5866.

In another aspect, the invention features a second method for assigning whether a sample includes STEC, the method includes the steps of: (a) providing nucleic acids from a sample; (b) detecting an O157:H7-specific fragment and a ECF-specific fragment; (c) assigning to the sample one of the following outcomes: 1) if the O157:H7-specific fragment and the ECF-specific fragment are absent then the sample is negative for O157:H7 STEC and a virulent non-O157:H7 STEC is present; 2) if the O157:H7-specific fragment is present and the ECF-specific fragment is absent then the sample is negative for a virulent non-O157:H7 STEC; 3) if the O157:H7-specific fragment and the ECF-specific fragment are both present then the sample includes an O157:H7 STEC; or 4) if the O157:H7-specific fragment is absent and the ECF-specific fragment is present then the sample includes a virulent non-O157:H7 STEC. Exemplary O157:H7-specific fragments include katP junction or Z5866 as is described herein. Exemplary virulent, non-O157:H7 STEC includes O26, O45, O103, O111, O121, or O145. The method also involves, in certain embodiments, detection of at least two O157:H7-specific fragments.

In another aspect, the invention features a third method of assigning whether a sample includes STEC, the method includes the steps of: (a) providing nucleic acids from a sample; (b) detecting a first fragment that detects O157 STEC and STEC lacking an ECF gene, and a second fragment that detects an ECF gene; (c) assigning to the sample one of the following outcomes: 1) if the first and second fragments are absent then the sample is negative for virulent O157 STEC and a virulent non-O157:H7 STEC; 2) if the first fragment is present and the second fragment is absent then the sample is negative for a virulent non-O157:H7 STEC; 3) if the first fragment and second fragment are present then the sample includes virulent O157 STEC; or 4) if the first fragment is absent and the second fragment is present then the sample includes a virulent non-O157:H7 STEC. Exemplary first fragments include Sil or Z0372, as is described herein. Exemplary virulent O157 STEC includes O157:H7, O157:NM, O157:H-, O157:H8, or O157:H21. And exemplary virulent, non-O157:H7 STEC includes O26, O45, O103, O111, O121, or O145. The method also involves detection of at least two first fragments (e.g., Sil and Z0372).

In another aspect, the invention features a fourth method of assigning whether a sample includes STEC, the method includes the steps of: (a) providing nucleic acids from a sample; (b) detecting a first fragment that detects O157:H7 STEC and STEC lacking an ECF gene, and a second fragment that detects the ECF gene; (c) assigning to the sample one of the following outcomes: 1) if the first and second fragments are absent then the sample is negative for O157:H7 STEC and a virulent non-O157:H7 STEC; 2) if the first fragment is present and the second fragment is absent then the sample is negative for virulent non-O157:H7 STEC; 3) if the first fragment and second fragment are present then the sample includes an O157:H7 STEC; or 4) if the first fragment is absent and the second fragment is present then the sample includes a virulent non-O157:H7 STEC. Exemplary virulent, non-O157:H7 STEC include O26, O45, O103, O111, O121, or O145.

In another aspect, the invention features still a method for detecting STEC in a sample, the method including the steps of: a) providing a sample including nucleic acid molecules; b) contacting the nucleic acid molecules with a virulent O157 STEC-specific probe and an ECF-specific probe under hybridization conditions, wherein i) the virulent O157 STEC-specific probe specifically hybridizes to a virulent O157 STEC-specific fragment of the nucleic acid molecules; and ii) the ECF-specific probe specifically hybridizes to an ECF-specific fragment of the nucleic acid molecules; and c) detecting hybridization of the virulent O157 STEC-specific probe and the ECF-specific probe to identify the presence or absence of the virulent O157 STEC-specific fragment or the ECF-specific fragment as an indication of the presence of absence of STEC in the sample. Typically, the absence of the virulent O157 STEC-specific fragment and absence of the ECF-specific fragment is taken as an indication that the sample is negative for virulent O157 STEC and a virulent non-O157:H7 STEC; the presence of the virulent O157-specific fragment and the absence of the ECF-specific fragment is taken as an indication that the sample is negative for a virulent non-O157:H7 STEC; the presence of the virulent O157-specific fragment and the presence of the ECF-specific fragment is taken as an indication that the sample is positive for virulent O157 STEC; or the absence of the virulent O157 STEC-specific fragment and the presence of the ECF-specific fragment is taken as an indication that the sample is positive for a virulent non-O157:H7 STEC. Exemplary virulent O157 STEC-specific fragments include rfb, wzx, or wzy. Exemplary virulent O157 STEC includes O157:H7, O157:NM, O157:H-, O157:H8, or O157:H21. And exemplary virulent, non-O157:H7 STEC includes O26, O45, O103, O111, O121, or O145. The method also involves detection of at least two virulent O157 STEC-specific fragments (e.g., rfb and wzk, rfb and wzy, and wzk and wzy, or rfb, wzk, and wzy). Exemplary methods for detecting hybridization involve amplification or cDNA synthesis. Nucleic acid molecules, if desired, are typically purified from an environmental or a biological sample (e.g., a food sample such as meat).

In another aspect, the invention features a method for detecting STEC in a sample, the method includes the steps of: a) providing a sample including nucleic acid molecules; b) contacting the nucleic acid molecules with an O157:H7-specific probe and an ECF-specific probe under hybridization conditions, wherein i) the O157:H7-specific probe specifically hybridizes to an O157:H7-specific fragment of the nucleic acid molecules; and ii) the ECF-specific probe specifically hybridizes to an ECF-specific fragment of the nucleic acid molecules; and c) detecting hybridization of the O157:H7-specific probe and the ECF-specific probe to identify the presence or absence of the O157:H7-specific fragment or the ECF-specific fragment as an indication of the presence of absence of STEC in the sample. Typically, the absence of the O157:H7-specific fragment and absence of the ECF-specific fragment is taken as an indication that the sample is negative for O157:H7 STEC and a virulent non-O157:H7 STEC; the presence of the O157:H7-specific fragment and the absence of the ECF-specific fragment is taken as an indication that the sample is negative for a virulent non-O157:H7 STEC; the presence of the O157:H7-specific fragment and the presence of the ECF-specific fragment is taken as an indication that the sample is positive for an O157:H7 STEC; or the absence of the O157:H7-specific fragment and the absence of the ECF-specific fragment is taken as an indication that the sample is positive for a virulent non-O157:H7 STEC. Exemplary O157:H7-specific fragments include katP junction or 25866 as is described herein. Exemplary virulent, non-O157:H7 STEC include O26, O45, O103, O111, O121, or O145. The method also involves detection of at least two O157:H7-specific fragments (e.g, katP and Z5866). Standard methods for detecting hybridization involve amplification or cDNA synthesis. Nucleic acid molecules, if desired, are typically purified from an environmental or a biological sample (e.g., a food sample such as meat).

In another aspect, the invention features a method for detecting STEC in a sample, the method includes the steps of: a) providing a sample including nucleic acid molecules; b) contacting the nucleic acid molecules with a first probe and a second probe under hybridization conditions, wherein i) the first probe specifically hybridizes with nucleic acid molecules of (1) a virulent O157 STEC and (2) STEC lacking an ECF gene; and ii) the second probe specifically hybridizes to an ECF-specific fragment of the nucleic acid molecules; and c) detecting hybridization of the first probe and the second probe, wherein the presence or absence of hybridization to the first probe and the second probe is taken as indication of the presence or absence of STEC in the sample. Typically, the absence of hybridization to the first probe and absence of hybridization to the second probe is taken as an indication that the sample is negative for virulent O157 STEC and a virulent non-O157:H7 STEC; the presence of hybridization to the first probe and the absence of hybridization to the second probe is taken as an indication that the sample is negative for a virulent non-O157:H7 STEC; the presence of hybridization to the first probe and the presence of hybridization to the second probe is taken as an indication that the sample is positive for virulent O157 STEC; or the absence of hybridization to the first probe and the presence of hybridization to the second probe is taken as an indication that the sample is positive for a virulent non-O157:H7 STEC. Exemplary first fragments include Sil or Z0372 as is described herein. Exemplary virulent O157 STEC includes O157:H7, O157:NM, O157:H-, O157:H8, or O157:H21. Exemplary virulent, non-O157:H7 STEC includes O26, O45, O103, O111, O121, or O145. The method also involves detection of at least two first fragments (e.g., Sil and Z0372). Standard methods for detecting hybridization involve amplification or cDNA synthesis. Nucleic acid molecules, if desired, are typically purified from an environmental or a biological sample (e.g. a food sample such as meat).

In still another aspect, the invention features an method for detecting STEC in a sample, the method including the steps of: a) providing a sample including nucleic acid molecules; b) contacting the nucleic acid molecules with a first probe and a second probe under hybridization conditions, wherein i) the first probe specifically hybridizes with nucleic acid molecules of (1) an O157:H7 STEC and (2) STEC lacking an ECF gene; and ii) the second probe specifically hybridizes to an ECF-specific fragment of the nucleic acid molecules; and c) detecting hybridization of the first probe and the second probe, wherein the presence or absence of hybridization to the first probe and the second probe is taken as indication of the presence or absence of STEC in the sample. Typically, the absence of hybridization to the first probe and absence of hybridization to the second probe is taken as an indication that the sample is negative for O157 STEC and a virulent non-O157:H7 STEC; the presence of hybridization to the first probe and the absence of hybridization to the second probe is taken as an indication that the sample is negative for a virulent non-O157:H7 STEC; the presence of hybridization to the first probe and the presence of hybridization to the second probe is taken as an indication that the sample is positive for an O157:H7 STEC; or the absence of hybridization to the first probe and the presence of hybridization to the second probe is taken as an indication that the sample is positive for a virulent non-O157:H7 STEC. Standard methods for detecting hybridization involve amplification or cDNA synthesis. Nucleic acid molecules, if desired, are typically purified from an environmental or a biological sample (e.g., a food sample such as meat).

In another aspect, the invention features a method for assessing the presence or absence of virulent non-O157:H7 STEC in a sample, the method includes the steps of: a) contacting nucleic acid molecules from the sample with an ECF-specific probe under hybridization conditions, wherein the ECF-specific probe specifically hybridizes to an ECF-specific region; and b) detecting hybridization of the ECF-specific probe and the nucleic acid molecules, wherein presence or absence of hybridization of the ECF-specific probe with the nucleic acid molecules indicates the presence or absence of virulent non-O157:H7 STEC in the sample. Typically, the nucleic acid molecules are contacted with a virulent O157 STEC-specific probe that specifically hybridizes to a virulent O157 STEC-specific fragment of the nucleic acid molecules, and wherein (i) absence of hybridization of the O157 STEC-specific probe and absence of hybridization of the ECF-specific probe is taken as an indication that the sample is negative for virulent O157 STEC and a virulent non-O157:H7 STEC; (ii) the presence of hybridization of the virulent 0157-specific fragment and the absence of hybridization of the ECF-specific fragment is taken as an indication that the sample is negative for a virulent non-O157:H7 STEC; (iii) the presence of hybridization of the virulent O157-specific fragment and the presence of hybridization of the ECF-specific fragment is taken as an indication that the sample is positive for virulent O157 STEC; or (iv) the absence of hybridization of the virulent O157 STEC-specific fragment and the presence of hybridization of the ECF-specific fragment is taken as an indication that the sample is positive for a virulent non-O157:H7 STEC. The nucleic acid molecules may also be contacted with a O157:H7-specific probe that specifically hybridizes to an O157:H7-specific fragment of the nucleic acid molecules, and (i) the absence of hybridization of the O157:H7-specific fragment and absence of hybridization of the ECF-specific fragment is taken as an indication that the sample is negative for O157:H7 STEC and a virulent non-O157:H7 STEC; (ii) the presence of hybridization of the O157:H7-specific fragment and the absence of hybridization of the ECF-specific fragment is taken as an indication that the sample is negative for a virulent non-O157:H7 STEC; (iii) the presence of hybridization of the O157:H7-specific fragment and the presence of hybridization of the ECF-specific fragment is taken as an indication that the sample is positive for an O157:H7 STEC; and (iv) the absence of hybridization of the O157:H7-specific fragment and the absence of the ECF-specific fragment is taken as an indication that the sample is positive for a virulent non-O157:H7 STEC.

Similarly, the nucleic acid molecules may be contacted with a probe (a′) that specifically hybridizes with nucleic acid molecules of (1) a virulent O157 STEC and (2) STEC lacking an ECF gene; and wherein (i) the absence of hybridization to the probe (a′) and absence of hybridization to the ECF-specific fragment is taken as an indication that the sample is negative for virulent O157 STEC and a virulent non-O157:H7 STEC, (ii) the presence of hybridization to the probe (a′) and the absence of hybridization to the ECF-specific fragment is taken as an indication that the sample is negative for a virulent non-O157:H7 STEC; (iii) the presence of hybridization to the probe (a′) and the presence of hybridization to the ECF-specific fragment is taken as an indication that the sample is positive for virulent O157 STEC, (iv) the absence of hybridization to the probe (a′) and the presence of hybridization to the ECF-specific fragment is taken as an indication that the sample is positive for a virulent non-O157:H7 STEC.

And, if desired, the nucleic acid molecules may be contacted with a probe (b′) that specifically hybridizes with nucleic acid molecules of (1) an O157:H7 STEC and (2) STEC lacking an ECF gene, and wherein (i) the absence of hybridization to probe (b′) and absence of hybridization to the ECF-specific fragment is taken as an indication that the sample is negative for O157 STEC and a virulent non-O157:H7 STEC; (ii) the presence of hybridization to the probe (b′) and the absence of hybridization to the ECF-specific fragment is taken as an indication that the sample is negative for a virulent non-O157:H7 STEC, (iii) the presence of hybridization to the probe (b′) and the presence of hybridization to the ECF-specific fragment is taken as an indication that the sample is positive for an O157:H7 STEC, and (iv) the absence of hybridization to the probe (b′) and the presence of hybridization to the ECF-specific fragment is taken as an indication that the sample is positive for a virulent non-O157:H7 STEC.

In still another number of aspects, the invention features targets for identifying a STEC as well as oligonucleotides or primers, alone or in combination, which are useful for identifying or amplifying such targets. Exemplary target sequences and oligonucleotides are described herein (see, for example, FIGS. 1-9 and Table 2, respectively).

Accordingly, in another aspect, the invention features a nucleic acid consisting of a nucleic acid sequence wherein the nucleic acid sequence is a 1318 bp Z5886 shown in FIG. 1 or a fragment thereof or sequence complementary thereto.

In another aspect, the invention features a composition including a nucleic acid consisting of a nucleic acid sequence wherein the nucleic acid sequence is a fragment of the Ecf gene cluster shown in FIG. 2 or a fragment thereof or sequence complementary thereto, wherein the fragment is 1-2404 bp or 3584-5612 bp as shown in FIG. 2. Exemplary nucleic acid sequences are the 949 bp Ecf2-1 fragment or the 1050 bp Ecf2-2 fragment, each disclosed herein. For example, an isolated nucleic acid sequence selected from the group consisting of: 5′-CCC TTA TGA AGA GCC AGT ACT GAA G-3′ (SEQ ID NO: 1) and 5′ ATT ACG CAT AGG GCG TAT CAG CAC-3′ (SKI ID NO: 2).

Other Ecf primers include the following or combinations thereof:

| | | SEQ ID |
|---|---|---|
| | sequence | NO: |
| ecf1 Set 1 | CCC TTA TGA AGA GCC AGT ACT | 1 |
| Forward | GAA G | |
| Primer | | |
| ecf1 Set 1 | ATT ACG CAT AGG GCG TAT CAG | 2 |
| Reverse | CAC | |
| Primer | | |
| ecf1 Set 3 | TGC AAG GCA TCT TCC CGT ACT | 3 |
| Forward | GAT | |
| Primer | | |
| ecf1 Set 3 | TCT GCG AGC CAC TTC ATC TGT | 4 |
| Reverse | TCA | |
| Primer | | |
| ecf1 Set 5 | AGC AGG AAT ATT CTC ACC GCG | 5 |
| Forward | ACT | |
| Primer | | |
| ecf1 Set 5 | ACA GAC AAC CTG TCC CAG CGT | 6 |
| Reverse | TTA | |
| Primer | | |
| ecf3 Set 1 | TTC CTT TGC CAT GGC GGA GAA | 7 |
| Forward | TTG | |
| Primer | | |
| ecf3 Set 1 | AGC GGC TCC TGT CTG ATT AAC | 8 |
| Reverse | GAT | |
| Primer | | |
| ecf3 Set 4 | TGA TCA TCG TGC ATC TGC TGG | 9 |
| Forward | GTA | |
| Primer | | |
| ecf3 Set 4 | ATG CCC TGT AAT GCC ATC AAA | 10 |
| Reverse | CCG | |
| Primer | | |
| ecf3 Set 5 | TGT ACA CTG TTC CGT TCC TGC | 11 |
| Forward | TGT | |
| Primer | | |
| ecf3 Set 5 | TCC CTG AAT TGC GGA TTC ACC | 12 |
| Reverse | AGA | |
| Primer | | |
| ecf4 Set 3 | ACG CTG GAA TGG TCT GGA GAT | 13 |
| Forward | TGT | |
| Primer | | |
| ecf4 Set 3 | ATC CAC CAC CGG ATT TCT CTG | 14 |
| Reverse | GTT | |
| Primer | | |
| ecf4 Set 4 | AAC TTT ACC GGT TAT CGG ACG | 15 |
| Forward | GCT | |
| Primer | | |
| ecf4 Set 4 | TGC TCA GGA TGT GGA CGA ACG | 16 |
| Reverse | AAA | |
| Primer | | |
| ecf4 Set 1 | TGG TAC CAC CTT CTG CTG TAC | 17 |
| Forward | TCT | |
| Primer | | |
| ecf4 Set 1 | TAC CTG TCC ACG TCA TCC AGT | 18 |
| Reverse | AAC | |
| Primer | | |

In still another aspect, the invention features a composition including a nucleic acid consisting of a nucleic acid sequence wherein the nucleic acid sequence is a 1269 bp Rfb_{O157} shown in FIG. 3 or a fragment thereof or sequence complementary thereto.

In another aspect, the invention features a composition including a nucleic acid consisting of a nucleic acid sequence wherein the nucleic acid sequence is a 1392 bp Wzx_{O157} shown in FIG. 4 or a fragment thereof or sequence complementary thereto.

In another aspect, the invention features a composition including a nucleic acid consisting of a nucleic acid sequence wherein the nucleic acid sequence is a 1185 bp Wzy_{O157} shown in FIG. 5 or a fragment thereof or sequence complementary thereto.

In yet another aspect, the invention features a composition including a nucleic acid consisting of a nucleic acid sequence wherein the nucleic acid sequence is a 2634 bp SIL_{O157} shown in FIG. 6 or a fragment thereof or sequence complementary thereto.

In another aspect, the invention features a composition including a nucleic acid consisting of a nucleic acid sequence wherein the nucleic acid sequence is a 279 bp Z0344 shown in FIG. 7 or a fragment thereof or sequence complementary thereto.

And in another aspect, the invention features a composition including a nucleic acid consisting of a nucleic acid sequence wherein the nucleic acid sequence is a 357 bp Z0372 shown in FIG. 8 or a fragment thereof or sequence complementary thereto.

The invention also features oligonucleotides that bind to any of the aforementioned targets as well as combinations of any of these oligonucleoetides.

Accordingly, the invention further features a composition, including: a first oligonucleotide that has a target-complementary base sequence to Ecf2-1 or Ecf2-2, optionally including a 5′ sequence that is not complementary to the specific target sequence.

In addition, the invention features a composition, including: a first oligonucleotide that has a target-complementary base sequence to Ecf gene cluster, optionally including a 5′ sequence that is not complementary to the specific target sequence and a second oligonucleotide. Exemplary second oligonucleotides include, without limitation, an oligonucleotide selected from the group consisting of:
a.) an oligonucleotide that has a target-complementary base sequence to Z5886, optionally including a 5′ sequence that is not complementary to the specific target sequence;
b.) an oligonucleotide that has a target-complementary base sequence to hylA, optionally including a 5′ sequence that is not complementary to the specific target sequence;
c.) an oligonucleotide that has a target-complementary base sequence to rfb_{O157}, optionally including a 5′ sequence that is not complementary to the specific target sequence;
d.) an oligonucleotide that has a target-complementary base sequence to wzx_{O157}, optionally including a 5′ sequence that is not complementary to the specific target sequence;
e.) an oligonucleotide that has a target-complementary base sequence to wzy_{O157}, optionally including a 5′ sequence that is not complementary to the specific target sequence;
f.) an oligonucleotide that has a target-complementary base sequence to SIL_{O157}, optionally including a 5′ sequence that is not complementary to the specific target sequence.
g.) an oligonucleotide that has a target-complementary base sequence to Z0344, optionally including a 5′ sequence that is not complementary to the specific target sequence;
h.) an oligonucleotide that has a target-complementary base sequence to Z0372, optionally including a 5′ sequence that is not complementary to the specific target sequence;
i.) an oligonucleotide that has a target-complementary base sequence to katP junction, optionally including a 5′ sequence that is not complementary to the specific target sequence.

Such compositions are prepared, if desired, so that only one of the first and second oligonucleotides has a 3′ end that can be extended by a template-dependent DNA polymerase. Further, if desired, an oligonucleotide may include a detectably labeled hybridization probe.

The invention provides long awaited advantages over a wide variety of standard screening methods used for distinguishing and evaluating STEC. In particular, the invention disclosed herein reduces not only the number of false positives typically obtained when compared to current methods but also reduces the number of tests and steps performed on a sample. The invention accordingly obviates many issues encountered when analyzing a sample in which many microorganism co-infections result in a high false positive rate.

Accordingly, the methods of the invention provide a facile means to identify and distinguish STEC. In addition, the methods of the invention provide a route for analyzing virtually any number of samples for presence of STEC with high-volume throughput and high sensitivity. The methods are also relatively inexpensive to perform and enable the analysis of small quantities of samples found in either purified or crude extract form.

Further, the invention disclosed herein advantageously demonstrates specificity for distinguishing highly virulent non-O157:H7 STEC, including the big six non-O157:H7 STECs, from O157:H7.

Other features and advantages of the invention will be apparent from the following description of the preferred embodiments thereof, and from the claims.

### BRIEF DESCRIPTION OF THE DRAWINGS

FIG. 1 shows a 1318 bp sequence of Z5886. Forward and reverse primers used to generate an 80 bp amplicon are also shown.

FIG. 2 shows a 5612 bp sequence of the ECF gene cluster as well as Ecf2-1 and Ecf2-2 fragments respectively 949 bp and 1050 bp. Forward and reverse primers used to generate a 114 bp amplicon are also shown in connection with the ECF gene cluster and Ecf2-1 gene fragment.

FIG. 3 shows a 1269 bp sequence of Rfb_{O157}. Forward and reverse primers used to generate a 141 bp amplicon are also shown.

FIG. 4 shows a 1392 bp sequence of wzx_{O157}. Forward and reverse primers used to generate a 122 bp amplicon are also shown. Forward and reverse primers used to generate a 167 bp amplicon are shown as well.

FIG. 5 shows a 1185 bp sequence of wzy. Forward and reverse primers used to generate a 191 bp amplicon are also shown.

FIG. 6 shows a 2634 bp sequence of SIL_{O157}. Forward and reverse primers used to generate a 152 bp amplicon are shown.

FIG. 7 shows a 279 bp sequence of Z0344. Forward and reverse primers used to generate a 125 bp amplicon are shown.

FIG. 8 shows a 357 bp sequence of Z0372. Forward and reverse primers used to generate a 177 bp amplicon are shown.

FIG. 9 shows a 1489 bp sequence of katP junction. Forward and reverse primers used to generate a 101 bp amplicon are shown.

FIG. 10 shows polymerase chain reaction (PCR) screening results testing 214 E. coli strains for identifying virulent O157:H7 and non-O157 STEC.

### DETAILED DESCRIPTION OF THE INVENTION

In certain aspects and embodiments, the invention relates to compositions, methods and kits for the identification, detection, and/or quantitation of E. coli STEC, which may be present either alone or as a component, large or small, of a homogeneous or heterogeneous mixture of nucleic acids in a sample taken for testing, e.g., for diagnostic testing, for screening of blood products, for microbiological detection in bioprocesses, food such as meat or dairy products, water, animals such as reservoirs of O157:H7 and non-O157:H7 STEC such as ruminants and other animals, industrial or environmental samples, and for other purposes. Specific methods, compositions, and kits as disclosed herein provide improved sensitivity, specificity, or speed of detection in the amplification-based detection of E. coli STEC such as O157:H7 and non-O157:H7 STEC. Accordingly, in certain embodiments of the invention, assays disclosed herein identify ecf sequences common to E. coli O157:H7 and non-O157:H7 STEC, and differentiates E. coli STECs including virulent non-O157 STECs such as O26, O45, O103, O111, O121, and O145 from other non-virulent strains and, for example, from O157:H7. A preferred useful region for such differentiation is the ECF gene cluster, for example Ecf2-1 and Ecf2-2.

As a result of extensive analyses of amplification oligonucleotides specific for E. coli O157:H7, the particular region of E. coli O157:H7, corresponding to the region of E. coli Ecf2-1 sequence, has been identified as a target for amplification-based detection of E. coli O157:H7 and non-O157:H7 STEC. In addition, after extensive analysis a particular region of E. coli O157:H7 (Z5886)(hereinafter referred to as the “Z5886 region”) has been identified as still another useful target for amplification-based detection of E. coli O157:H7. Other useful regions include rfb_{O157}, wzx_{O157}, wzy_{O157}, Z0344, Z0372, SIL_{O157}, and katP junction as is disclosed herein. Accordingly, the invention relates to methods of detection of E. coli O157:H7 and non-O157:H7 STEC in a sample of interest, amplification oligonucleotides, compositions, reaction mixtures, and kits.

The assays described herein detect sequences specific for STEC from other non-virulent strains. The assays also provide for the detection of the big six virulent, non-O157:H7 STEC. It may utilize virtually any known nucleic amplification protocol such as real-time polymerase chain reaction (PCR) or real-time transcription mediated amplification (TMA), where the target-specific sequence is amplified and a fluorescent molecular torch is used to detect the amplified products as they are produced. Target detection is performed simultaneously with the amplification and detection of an internal control in order to confirm reliability of the result. The result of the assay consists of the classification of the sample as positive or negative for the presence or absence of STEC.

In one embodiment, the sample is a blood sample or a contaminated meat product where STEC is a known or suspected contaminant. Using the methods disclosed herein, for example, the presence of STEC in one or more contaminated samples may be monitored in a rapid and sensitive fashion.

Target Nucleic Acid/Target Sequence

Target nucleic acids may be isolated from any number of sources based on the purpose of the amplification assay being carried out. The present invention provides a method for detecting and distinguishing between E. coli (e.g., O157 STEC and virulent non-O157 strains) using a hybridization assay that may also include a nucleic amplification step that precedes a hybridization step. Preparation of samples for amplification of E. coli sequences may include separating and/or concentrating organisms contained in a sample from other sample components according to standard techniques, e.g., filtration of particulate matter from air, water, or other types of samples. Once separated or concentrated, the target nucleic acid may be obtained from any medium of interest, such as those described above and, in particular, contaminated food. Sample preparation may also include chemical, mechanical, and/or enzymatic disruption of cells to release intracellular contents, including E. coil RNA or DNA. Preferred samples are food and environmental samples. Methods to prepare target nucleic acids from various sources for amplification are well known to those of ordinary skill in the art. Target nucleic acids may be purified to some degree prior to the amplification reactions described herein, but in other cases, the sample is added to the amplification reaction without any further manipulations.

Sample preparation may include a step of target capture to specifically or non-specifically separate the target nucleic acids from other sample components. Nonspecific target preparation methods may selectively precipitate nucleic acids from a substantially aqueous mixture, adhere nucleic acids to a support that is washed to remove other sample components, or use other means to physically separate nucleic acids, including STEC nucleic acid, from a mixture that contains other components. Other nonspecific target preparation methods may selectively separate RNA from DNA in a sample.

A target sequence may be of any practical length. An optimal length of a target sequence depends on a number of considerations, for example, the amount of secondary structure, or self-hybridizing regions in the sequence. Typically, target sequences range from about 30 nucleotides in length to about 300 nucleotides in length or greater. Target sequences accordingly may range from 3-100, 50-150, 75-200, 100-500, or even 500-800 or 900-1,100 nucleotides in length. The optimal or preferred length may vary under different conditions which can be determined according to the methods described herein and the sequences of the targets described herein.

Nucleic Acid Identity

In some instances, a nucleic acid comprises a contiguous base region that is at least 70%; or 75%; or 80%, or 85% or 90%, or 95%, or even 96%, 97%, 98%, 99% or even 100% identical to a contiguous base region of a reference nucleic acid. For short nucleic acids, the degree of identity between a base region of a query nucleic acid and a base region of a reference nucleic acid can be determined by manual alignment or using any standard alignment tool known in the art such as “BLAST.” “Identity’ is simply determined by comparing just the nucleic acid sequences. Thus, the query:reference base sequence alignment may be DNA:DNA, RNA:RNA, DNA:RNA, RNA:DNA, or any combinations or analogs thereof. Equivalent RNA and DNA base sequences can be compared by converting U's (in RNA) to T's (in DNA).

Oligonucleotides

An oligonucleotide can be virtually any length, limited only by its specific function in the amplification reaction or in detecting an amplification product of the amplification reaction. However, in certain embodiments, preferred oligonucleotides will contain at least about 5, 6, 7, 8, 9, or 10; or 11, 12, 13, 14, 15, 16, 17, 18, 19 or 20; or 22; or 24; or 26; or 28; or 30; or 32; or 34; or 36; or 38; or 40; or 42; or 44; or 46; or 48; or 50; or 52; or 54; or 56 contiguous bases that are complementary to a region of the target nucleic acid sequence or its complementary strand. The contiguous bases are preferably at least about 80%, more preferably at least about 90%, and most preferably completely complementary to the target sequence to which the oligonucleotide binds. Certain preferred oligonucleotides are of lengths generally between about 5-20, 5-25, 10-100; or 12-75; or 14-50; or 15-40 bases long and optionally can include modified nucleotides. Exemplary oligonucleotides are described herein.

Oligonucleotides may be modified in any way, as long as a given modification is compatible with the desired function of a given oligonucleotide. One of ordinary skill in the art can easily determine whether a given modification is suitable or desired for any given oligonucleotide. Modifications include base modifications, sugar modifications or backbone modifications.

Nucleic Acid Amplification

Many well-known methods of nucleic acid amplification require thermocycling to alternately denature double-stranded nucleic acids and hybridize primers; however, other well-known methods of nucleic acid amplification are isothermal. Exemplary amplification methods include polymerase chain reaction (“PCR”), the ligase chain reaction (“LCR”), strand displacement amplification (“SDA”), nucleic acid sequence based amplification (“NASBA”), self-sustained sequence replication, and transcription-mediated amplification (“TMA”).

Suitable amplification conditions can be readily determined by a skilled artisan in view of the present disclosure. Amplification conditions, as disclosed herein, refer to conditions which permit nucleic acid amplification. Amplification conditions may, in some embodiments, be less stringent than “stringent hybridization conditions” as described herein. By “stringent hybridization conditions” is meant hybridization assay conditions wherein a specific detection probe is able to hybridize with target nucleic acids over other nucleic acids present in the test sample. It will be appreciated that these conditions may vary depending upon factors including the GC content and length of the probe, the hybridization temperature, the composition of the hybridization reagent or solution, and the degree of hybridization specificity sought. Specific stringent hybridization conditions are disclosed herein.

Oligonucleotides used in the amplification reactions as disclosed herein may be specific for and hybridize to their intended targets under amplification conditions, but in certain embodiments may or may not hybridize under more stringent hybridization conditions. On the other hand, detection probes generally hybridize under stringent hybridization conditions. While the Examples section infra provides preferred amplification conditions for amplifying target nucleic acid sequences, other acceptable conditions to carry out nucleic acid amplifications could be easily ascertained by someone having ordinary skill in the art depending on the particular method of amplification employed.

In a preferred embodiment, the target nucleic acid of a STEC can also be amplified by a transcription-based amplification technique. As is discussed above, one transcription-based amplification system is transcription-mediated amplification (TMA), which employs an RNA polymerase to produce multiple RNA transcripts of a target region. Exemplary TMA amplification methods are described in, e.g., U.S. Pat. Nos. 4,868,105; 5,124,246; 5,130,238; 5,399,491; 5,437,990; 5,480,784; 5,554,516; and 7,374,885; and PCT Pub. Nos. WO 88/01302; WO 88/10315 and WO 95/03430.

The methods of the present invention may include a TMA reaction that involves the use of a single primer TMA reaction, as is described in U.S. Pat. No. 7,374,885. In general, the single-primer TMA methods use a primer oligomer (e.g., a NT7 primer), a modified promoter-based oligomer (or “promoter-provider oligomer”; e.g., a T7 provider) that is modified to prevent the initiation of DNA synthesis from its 3′ end (e.g., by including a 3′-blocking moiety) and, optionally, a blocker oligomer (e.g., a blocker) to terminate elongation of a cDNA from the target strand. Promoter-based oligomers provide an oligonucleotide sequence that is recognized by an RNA polymerase. This single primer TMA method synthesizes multiple copies of a target sequence and includes the steps of treating a target RNA that contains a target sequence with a priming oligomer and a binding molecule, where the primer hybridizes to the 3′ end of the target strand. RT initiates primer extension from the 3′ end of the primer to produce a cDNA which is in a duplex with the target strand (e.g., RNA:cDNA). When a blocker oligomer, is used in the reaction, it binds to the target nucleic acid adjacent near the user designated 5′ end of the target sequence. When the primer is extended by DNA polymerase activity of RT to produce cDNA, the 3′ end of the cDNA is determined by the position of the blocker oligomer because polymerization stops when the primer extension product reaches the binding molecule bound to the target strand. Thus, the 3′ end of the cDNA is complementary to the 5′ end of the target sequence. The RNA:cDNA duplex is separated when RNase (e.g., RNase H of RT) degrades the RNA strand, although those skilled in the art will appreciate that any form of strand separation may be used. Then, the promoter-provider oligomer hybridizes to the cDNA near the 3′ end of the cDNA strand.

The promoter-provider oligomer includes a 5′ promoter sequence for an RNA polymerase and a 3′ target hybridizing region complementary to a sequence in the 3′ region of the cDNA. The promoter-provider oligomer also has a modified 3′ end that includes a blocking moiety that prevents initiation of DNA synthesis from the 3′ end of the promoter-provider oligomer. In the promoter-provider:cDNA duplex, the 3′-end of the cDNA is extended by DNA polymerase activity of RT using the promoter oligomer as a template to add a promoter sequence to the cDNA and create a functional double-stranded promoter.

An RNA polymerase specific for the promoter sequence then binds to the functional promoter and transcribes multiple RNA transcripts complementary to the cDNA and substantially identical to the target region sequence that was amplified from the initial target strand. The resulting amplified RNA can then cycle through the process again by binding the primer and serving as a template for further cDNA production, ultimately producing many amplicons from the initial target nucleic acid present in the sample. Some embodiments of the single-primer transcription-associated amplification method do not include the blocking oligomer and, therefore, the cDNA product made from the primer has an indeterminate 3′ end, but the amplification steps proceed substantially as described above for all other steps.

The methods of the invention may also utilize a reverse transcription-mediated amplification (RTMA), various aspects of which are disclosed in, e.g., U.S. Pat. Appln. Pub. No. US 2006-0046265 A1. RTMA is an RNA transcription-mediated amplification system using two enzymes to drive the reaction: RNA polymerase and reverse transcriptase. RTMA is isothermal; the entire reaction is performed at the same temperature in a water bath or heat block. This is in contrast to other amplification reactions such as PCR that require a thermal cycler instrument to rapidly change the temperature to drive reaction. RTMA can amplify either DNA or RNA, and can produce either DNA or RNA amplicons, in contrast to most other nucleic acid amplification methods that only produce DNA. RTMA has very rapid kinetics, resulting in a billion-fold amplification within 15-60 minutes. RTMA can be combined with a Hybridization Protection Assay (HPA), which uses a specific oligonucleotide probe labeled with an acridinium ester detector molecule that emits a chemiluminescent signal, for endpoint detection or with molecular torches for real-time detection. There are no wash steps, and no amplicon is ever transferred out of the tube, which simplifies the procedure and reduces the potential for contamination. Thus, the advantages of RTMA include amplification of multiple targets, results can be qualitative or quantitative, no transfers and no wash steps necessary, and detection can be in real time using molecular torches.

As an illustrative embodiment, the RTMA reaction is initiated by treating an RNA target sequence in a nucleic acid sample with both a tagged amplification oligomer and, optionally a blocking oligomer. The tagged amplification oligomer includes a target hybridizing region that hybridizes to a 3′-end of the target sequence and a tag region situated 5′ to the target hybridizing region. The blocking oligomer hybridizes to a target nucleic acid containing the target sequence in the vicinity of the 5′-end of the target sequence. Thus, the target nucleic acid forms a stable complex with the tagged amplification oligomer at the 3′-end of the target sequence and the terminating oligonucleotide located adjacent to or near the determined 5′-end of the target sequence prior to initiating a primer extension reaction. Unhybridized tagged amplification oligomers are then made unavailable for hybridization to a target sequence prior to initiating a primer extension reaction with the tagged priming oligonucleotide, preferably by inactivating and/or removing the unhybridized tagged priming oligonucleotide from the nucleic acid sample. Unhybridized tagged amplification oligomer that has been inactivated or removed from the system is then unavailable for unwanted hybridization to contaminating nucleic acids. In one example of removing unhybridized tagged amplification oligomer from a reaction mixture, the tagged amplification oligomer is hybridized to the target nucleic acid, and the tagged amplification oligomer:target nucleic acid complex is removed from the unhybridized tagged amplification oligomer using a wash step. In this example, the tagged amplification oligomer:target nucleic acid complex may be further complexed to a target capture oligomer and a solid support. In one example of inactivating the unhybridized tagged amplification oligomer, the tagged amplification oligomers further comprise a target-closing region. In this example, the target hybridizing region of the tagged amplification oligomer hybridizes to target nucleic acid under a first set of conditions (e.g., stringency). Following the formation of the tagged amplification oligomer:target nucleic acid complex the unhybridized tagged amplification oligomer is inactivated under a second set of the conditions, thereby hybridizing the target closing region to the target hybridizing region of the unhybridized tagged amplification oligomer. The inactivated tagged amplification oligomer is then unavailable for hybridizing to contaminating nucleic acids. A wash step may also be included to remove the inactivated tagged amplification oligomers from the assay.

An extension reaction is then initiated from the 3′-end of the tagged amplification oligomer with a DNA polymerase, e.g., reverse transcriptase, to produce an initial amplification product that includes the tag sequence. The initial amplification product is then separated from the target sequence using an enzyme that selectively degrades the target sequence (e.g., RNAse H activity). Next, the initial amplification product is treated with a promoter-based oligomer having a target hybridizing region and an RNA polymerase promoter region situated 5′ to the target hybridizing region, thereby forming a promoter-based oligomer:initial amplification product hybrid. The promoter-based oligomer may be modified to prevent the initiation of DNA synthesis, preferably by situating a blocking moiety at the 3′-end of the promoter-based oligomer (e.g., nucleotide sequence having a 3′-to-5′ orientation). The 3′-end of the initial amplification product is then extended to add a sequence complementary to the promoter, resulting in the formation of a double-stranded promoter sequence. Multiple copies of a RNA product complementary to at least a portion of the initial amplification product are then transcribed using an RNA polymerase, which recognizes the double-stranded promoter and initiates transcription therefrom. As a result, the nucleotide sequence of the RNA product is substantially identical to the nucleotide sequence of the target nucleic acid and to the complement of the nucleotide sequence of the tag sequence.

The RNA products may then be treated with a tag-targeting oligomer, which hybridizes to the complement of the tag sequence to form a tag-targeting oligomer: RNA product hybrid, and the 3′-end of the tag-targeting oligomer is extended with the DNA polymerase to produce an amplification product complementary to the RNA product. The DNA strand of this amplification product is then separated from the RNA strand of this amplification product using an enzyme that selectively degrades the first RNA product (e.g., RNAse H activity). The DNA strand of the amplification product is treated with the promoter-based oligomer, which hybridizes to the 3′-end of the second DNA primer extension product to form a promoter-based oligomer:amplification product hybrid. The promoter-based oligomer:amplification product hybrid then re-enters the amplification cycle, where transcription is initiated from the double-stranded promoter and the cycle continues, thereby providing amplification product of the target sequence.

Amplification product can then be used in a subsequent assay. One subsequent assay includes nucleic acid detection, preferably nucleic acid probe-based nucleic acid detection. The detection step may be performed using any of a variety of known ways to detect a signal specifically associated with the amplified target sequence, such as by hybridizing the amplification product with a labeled probe and detecting a signal resulting from the labeled probe. The detection step may also provide additional information on the amplified sequence, such as all or a portion of its nucleic acid base sequence. Detection may be performed after the amplification reaction is completed, or may be performed simultaneous with amplifying the target region, e.g., in real time. In one embodiment, the detection step allows detection of the hybridized probe without removal of unhybridized probe from the mixture (see, e.g., U.S. Pat. Nos. 5,639,604 and 5,283,174).

The amplification methods as disclosed herein, in certain embodiments, also preferably employ the use of one or more other types of oligonucleotides that are effective for improving the sensitivity, selectivity, efficiency, etc., of the amplification reaction.

Target Capture

At times, it may be preferred to purify or enrich a target nucleic acid from a sample prior to nucleic acid amplification. Target capture, in general, refers to capturing a target polynucleotide onto a solid support, such as magnetically attractable particles, wherein the solid support retains the target polynucleotide during one or more washing steps of the target polynucleotide purification procedure. In this way, the target polynucleotide is substantially purified prior to a subsequent nucleic acid amplification step. Many target capture methods are known in the art and suitable for use in conjunction with the methods described herein. For example, any support may be used, e.g., matrices or particles free in solution, which may be made of any of a variety of materials, e.g., nylon, nitrocellulose, glass, polyacrylate, mixed polymers, polystyrene, silane polypropylene, or metal. Illustrative examples use a support that is magnetically attractable particles, e.g., monodisperse paramagnetic beads to which an immobilized probe is joined directly (e.g., via covalent linkage, chelation, or ionic interaction) or indirectly (e.g., via a linker), where the joining is stable during nucleic acid hybridization conditions. In short, essentially any technique available to the skilled artisan may be used provided it is effective for purifying a target nucleic acid sequence of interest prior to amplification.

Nucleic Acid Detection

Any labeling or detection system or both used to monitor nucleic acid hybridization can be used to detect STEC amplicons. Such systems are well known in the art.

Detection systems typically employ a detection oligonucleotide of one type or another in order to facilitate detection of the target nucleic acid of interest. Detection may either be direct (i.e., probe hybridized directly to the target) or indirect (i.e., a probe hybridized to an intermediate structure that links the probe to the target). A probe's target sequence generally refers to the specific sequence within a larger sequence which the probe hybridizes specifically. A detection probe may include target-specific sequences and other sequences or structures that contribute to the probe's three-dimensional structure, depending on whether the target sequence is present

Essentially any of a number of well known labeling and detection system that can be used for monitoring specific nucleic acid hybridization can be used in conjunction with the present invention. Included among the collection of useful labels are fluorescent moieties (either alone or in combination with “quencher” moieties), chemiluminescent molecules, and redox-active moieties that are amenable to electronic detection methods. In some embodiments, preferred fluorescent labels include non-covalently binding labels (e.g., intercalating dyes) such as ethidium bromide, propidium bromide, chromomycin, acridine orange, and the like.

In some applications, probes exhibiting at least some degree of self-complementarity are desirable to facilitate detection of probe:target duplexes in a test sample without first requiring the removal of unhybridized probe prior to detection. By way of example, structures referred to as “molecular torches” and “molecular beacons” are designed to include distinct regions of self-complementarity and regions of target-complementarity. Molecular torches are fully described in U.S. Pat. Nos. 6,849,412, 6,835,542, 6,534,274, and 6,361,945, and molecular beacons are fully described in U.S. Pat. Nos. 5,118,801, 5,312,728, and 5,925,517.

Synthetic techniques and methods of attaching labels to nucleic acids and detecting labels are well known in the art.

Kits

The invention also features a kit for carrying out the described methods according to the present invention described herein. The kit includes nucleic acid probes or primers that may be labeled, reagents and containers for carrying out the hybridization assay, positive and negative control reagents, and instructions for performing the assay.

Some kits contain at least one target capture oligomer for hybridizing to a target nucleic acid. Some kits for detecting the presence or abundance of two or more target nucleic acids contain two or more target capture oligomers each configured to selectively hybridize to each of their respective target nucleic acids.

Some kits contain at least one first amplification oligomer for hybridizing to a target nucleic acid. Some kits for detecting the presence or abundance of two or more target nucleic acids contain two or more first amplification oligomers, each configured to selectively hybridize to their respective target nucleic acids.

Some kits contain chemical compounds used in performing the methods herein, such as enzymes, substrates, acids or bases to adjust pH of a mixture, salts, buffers, chelating agents, denaturants, sample preparation agents, sample storage or transport medium, cellular lysing agents, total RNA isolation components and reagents, partial generalized RNA isolation components and reagents, solid supports, and other inorganic or organic compounds. Kits may include any combination of the herein mentioned components and other components not mentioned herein. Components of the kits can be packaged in combination with each other, either as a mixture or in individual containers. It will be clear to skilled artisans that the invention includes many different kit configurations.

The kits of the invention may further include additional optional components useful for practicing the methods disclosed herein. Exemplary additional components include chemical-resistant disposal bags, tubes, diluent, gloves, scissors, marking pens, and eye protection.

### Example 1

We have developed a PCR to simultaneously detect E. coli O157:H7 and non-O157:H7 STEC, which provides sensitivity to identify non-O157:H7 STEC such as the big six virulent, non-O157:H7.

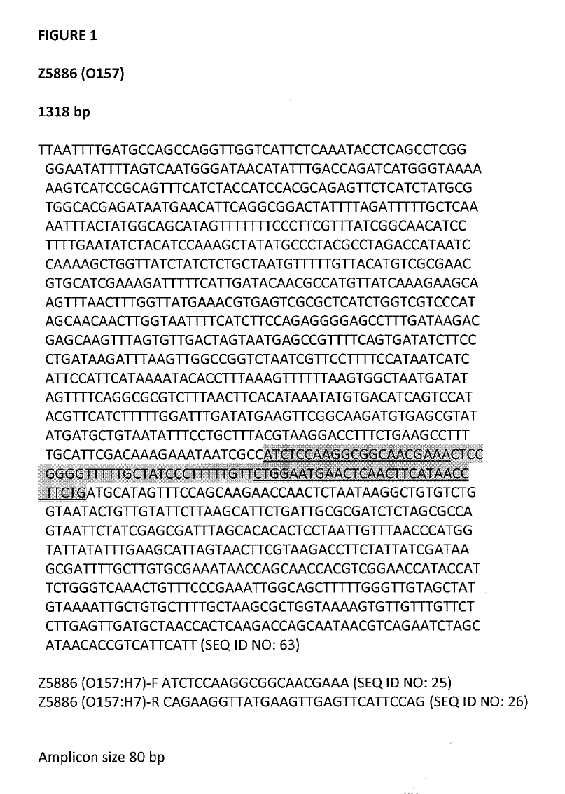

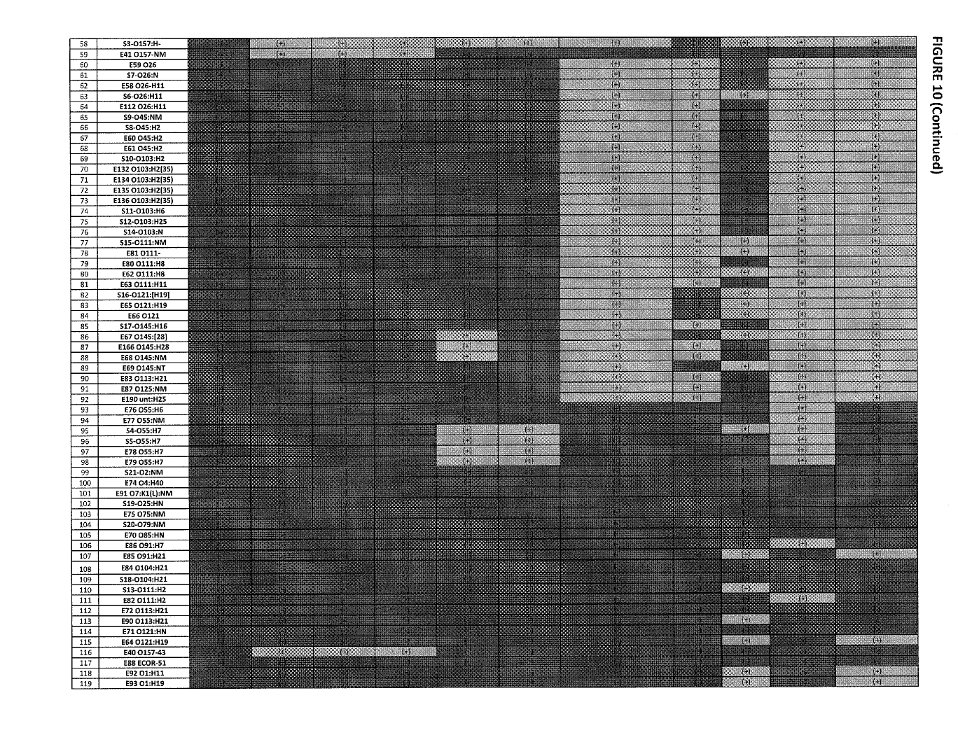

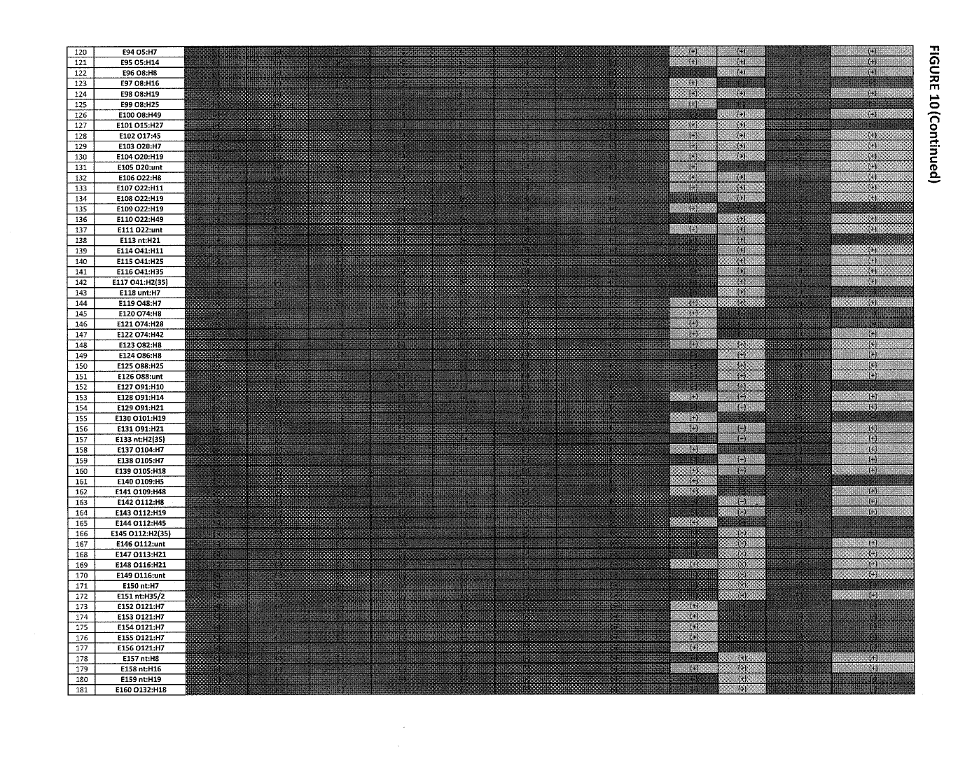

Useful targets identified for such assays include those found in FIGS. 1-9. Useful oligonucleotides for amplifying such targets are found in FIGS. 1-9 as well.

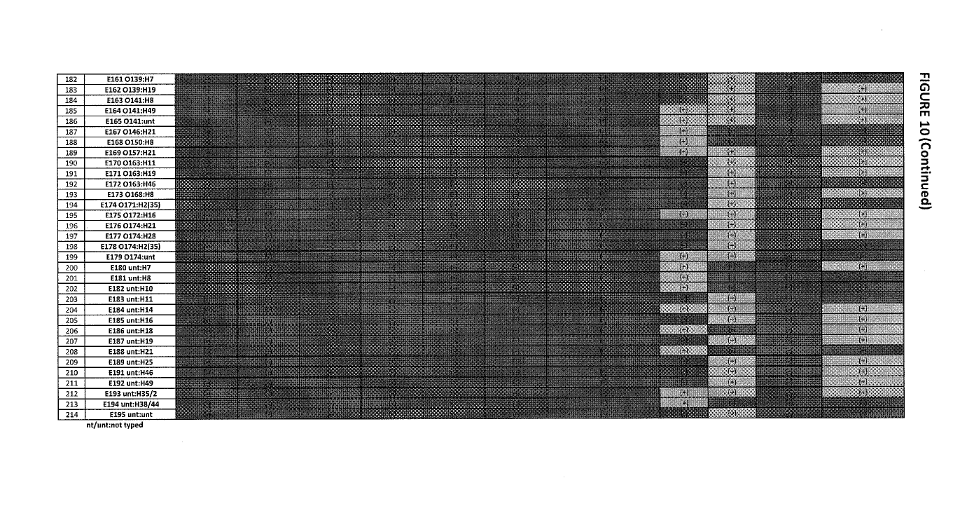

Accordingly, 214 E. coli strains shown in FIG. 10 were cultured according to standard methods. DNA was extracted from an overnight culture and purified using a PureLink Genomic DNA Kit (Invitrogen) according to kit instructions.

For sequencing, amplified DNA products were generated using a Clontech PCR kit consisting of the following master mix/reaction:

| Master Mix | Per Rxn |
|---|---|
| 10X Titanium Taq PCR Buffer | 6 uL |
| DNA template (100 ng/uL) | 3 uL |
| Primer Mix (10 uM each) | 2 uL |
| 50X dNTP mix (10 mM each of dATP, sCTP, dGTP, dTTP) | 1 uL |
| 50X Titanium Taq DNA Polymerase | 1 uL |
| Rnase-free H2O | 37 uL |
| Total Volume (per sample) | 50 uL |

Amplification conditions were as follows: 1 min at 95° C., 30 cycles of 30 seconds at 95° C. denature/90 seconds at 68° C. extension, followed by 90 seconds at 68° C. Amplified DNA was sequenced using oligos Z5866 F-1/Z5866 R-2 to detect target region Z5886 (O157:H7) and oligos ecf2-1 F/ecf2-1R and ecf2-2 F/ecf 2-2 R) to detect target regions ecf2-1 and ecf2-2 (STEC). Sequences of these primers are shown below in Table 1.

TABLE 1

| | | |
|---|---|---|
| Z5866 F-1 | 5′-TTA ATT TTG ATG CCA | (SEQ ID NO: 19) |
| | GCC AGG TTG G-3′ | |
| Z5866 R-2 | 5′-GCT AGA TTC TGA CGT | (SEQ ID NO: 20) |
| | TAT TGC TGG TC-3′ | |
| ecf2-1 F | 5′-AGG CAA GTA AAA CGG | (SEQ ID NO: 21) |
| | AAT GTC CCT GC-3′ | |
| ecf2-1 R | 5′-TAT GTT GAA TGC AAG | (SEQ ID NO: 22) |
| | GCA TCT TCC CG-3′ | |
| ecf2-2 F | 5′-GCT CTT TCG CAT TTA | (SEQ ID NO: 23) |
| | ATC CAG TGG GA-3′ | |
| ecf2-2 R | 5′-TAC AGC GGA ACG AAT | (SEQ ID NO: 24) |
| | GGA ATA CGG GA-3′ | |

Real Time PCR analysis was performed as follows. A real time master mix using the following ratio of components was prepared: 10 ul Power ABI SYBR Green Mixture/7.8 ul RNase-free H2O/0.2 ul Fwd/Rev primer. Primers are shown in Table 2. In a 96-well PCR plate, 2 ul of DNA template was added to 18 ul of real time master mix, sealed, and run on a Stratagene real time instrument using the following cycler conditions: denaturing for 10 minutes at 95° C., 40 cycles of 15 seconds at 95° C. denature/1 minute at 60° C. extension.

Replicates of each sample were run on Agilent Stratagene quantitative PCR machines for each respective primer pair and the data was subsequently compiled and analyzed using MxPro 3005P software.

TABLE 2

| | | |
|---|---|---|
| Z5886 | 5′-ATC TCC AAG GCG | (SEQ ID NO: 25) |
| (O157:H7)-F | GCA ACG AAA-3′ | |
| Z5886 | 5′-CAG AAG GTT ATG | (SEQ ID NO: 26) |
| (O157:H7)-R | AAG TTG AGT TCA TTC | |
| | CAG-3′ | |
| ecf (STEC)-F | 5′-CCC TTA TGA AGA | (SEQ ID NO: 1) |
| | GCC AGT ACT GAA G-3′ | |
| ecf (STEC)-R | 5′-ATT ACG CAT AGG | (SEQ ID NO: 2) |
| | GCG TAT CAG CAC-3′ | |
| stx1F | 5′-ATA AAT CGC CAT | (SEQ ID NO: 27) |
| | TCG TTG ACT AC-3′ | |
| stx1R | 5′-AGA ACG CCC ACT | (SEQ ID NO: 28) |
| | GAG ATC ATC-3′ | |
| stx2F | 5′-GGC ACT GTC TGA | (SEQ ID NO: 29) |
| | AAC TGC TCC-3′ | |
| stx2R | 5′-TCG CCA GTT ATC | (SEQ ID NO: 30) |
| | TGA CAT TCT G-3′ | |
| eaeSTEC-F | 5′-CAT TGA TCA GGA | (SEQ ID NO: 31) |
| | TTT TTC TGG TGA TA-3′ | |
| eaeSTEC-R | 5′-CTC ATG CGG AAA | (SEQ ID NO: 32) |
| | TAG CCG TTA-3′ | |
| rfbO157-F | 5′-CTGGACTCAACGTGGAT | (SEQ ID NO: 33) |
| | TTCATCA-3′ | |
| rfbO157-R | 5′-ACCTAACGCTAACAAAG | (SEQ ID NO: 34) |
| | CTAAATGAAG-3′ | |
| hlyASTEC-F | 5′-GTG TCA GTA GGG | (SEQ ID NO: 35) |
| | AAG CGA ACA-3′ | |
| hlyASTEC-R | 5′-ATC ATG TTT TCC | (SEQ ID NO: 36) |
| | GCC AAT G-3′ | |
| wzx1-F | 5′-TGC GTG TGG CAA | (SEQ ID NO: 37) |
| | AAA TTT AAA GAT-3′ | |
| wzx1-R | 5′-GTT GCC AAT CAA | (SEQ ID NO: 38) |
| | TCA TGC CAG AAG-3′ | |
| wzx2-F | 5′-AGT TAG GCA CTC | (SEQ ID NO: 39) |
| | TGG CAA CAT GGA-3′ | |
| wzx2-R | 5′-ATG AGC ATC TGC | (SEQ ID NO: 40) |
| | ATA AGC AGC CCA-3′ | |
| Z0344-F | 5′-CCT CTC AAT TGT | (SEQ ID NO: 41) |
| | CAG GGA AAT TAG | |
| | CGT-3′ | |
| Z0344-R | 5′-TGT TAA TGG TTG | (SEQ ID NO: 42) |
| | AAC CGA CGG CAG-3′ | |
| Z0372-F | 5′-GGA CGA CGA ATA | (SEQ ID NO: 43) |
| | AAT GTC ACT CCA CC-3′ | |
| Z0372-R | 5′-CAG CCT GGA TAC | (SEQ ID NO: 44) |
| | CGC TAC TCA AAT-3′ | |
| wzy-F | 5′-CAG TTA CTA CGT | (SEQ ID NO: 45) |
| | ATG GAG CAG AAC | |
| | TGT-3′ | |
| wzy-R | 5′-CGA TGC ATT CCC | (SEQ ID NO: 46) |
| | AGC CAC TAA GTA-3′ | |
| SIL-F | 5′-ATG AAT GCG CTG | (SEQ ID NO: 47) |
| | ACA ACC GAT GTG-3′ | |
| SIL-R | 5′-AAC TGT TGG TGC | (SEQ ID NO: 48) |
| | GTT TGG GTT ACG-3′ | |

Multiple E. coli STECs including O157:H7 and virulent non-O157 STECs such as O26, O45, O103, O111, O121, and O145 as well as non-virulent E. coli strains were tested. The data obtained from these PCR assays is summarized in FIG. 10. In particular, FIG. 10 shows PCR screening results testing 214 E. coli strains for specificity of O157:H7 (Z5886, rfb_{O157}, wzx_{O157}, Z0344, Z0372) and STEC (ecf) specific targets. In particular, these results show the specificity of the O157:H7 (Z5886, rfb_{O157}, wzx_{O157}, Z0344, Z0372) and STEC (ecf) specific targets, in addition to the genetic virulence profiles (stx1, stx2, eae, and hlyA). These data also demonstrate the specificity of O157 targets rfb_{O157}, wzx_{O157}, and Z0372 in combination with the ecf target region. The results also show that STEC (ecf) specific target detects only E. coli strains which have a combination of 3 virulence factors: stx1 or stx2 or stx1/stx2 in combination with eae_{STEC} and hlyA (ehx), and therefore is specific for highly virulent STEC/EHEC strains including the big six non-O157 serogroups—O26, O45, O103, O111, O121, and O145.

Further, we obtained 104 non-O157:H7 STEC isolates from the USDA (Bosilevac and Koohmaraie, Appl. Environ. Microbiol. 77(6):2103-2112, 2011). These isolates were tested with an O157:H7 specific target (Z5886), two O157 specific targets (rfb_{O157}, and wzx_{O157}), and an ecf fragment. As shown in Table 3 none of the non-O157:H7 STEC isolates were detected by the O157:H7 or O157 specific targets. Of the 104 non-O157:H7 STEC isolates, 6 were the so-called big six non-O157:H7 STEC isolates. These were detected by a PCR assay specific for the ecf fragment. One out of 104 non-O157 STEC isolates was detected by the ecf PCR assay but does not belong to the group of big six non-O157 STEC. This sample is a highly virulent EHEC/STEC isolate which contains three virulent markers, stx, eae and hlyA, and therefore is correctly detected by the ecf assay herein.

TABLE 3
Specificity of O157 and STEC target regions tested by real time
PCR (104 non-O157 STEC samples were tested).

| | | O157 | | | | | | STEC | |
|---|---|---|---|---|---|---|---|---|---|
| | | Z5886 | | rfb | | wzx | | ecf | |
| | n | pos | neg | pos | neg | pos | neg | pos | neg |
| O157:H7 | 0 | 0 | 0 | 0 | 0 | 0 | 0 | 0 | 0 |
| O157:NM | 0 | 0 | 0 | 0 | 0 | 0 | 0 | 0 | 0 |
| Top 6 non-O157 | 6 | 0 | 6 | 0 | 6 | 6 | 6 | 6 | 0 |
| STEC | | | | | | | | | |
| Non-top | 1 | 0 | 1 | 0 | 1 | 0 | 1 | 1 | 0 |
| 6 non-O157 | | | | | | | | | |
| STEC/EHEC | | | | | | | | | |
| Others | 97 | 0 | 97 | 0 | 97 | 0 | 97 | 0 | 97 |
| Total strains | 104 | | | | | | | | |
| tested | | | | | | | | | |

### Example 2

### Testing of E. Coli O157:H7, Stec Including Big 6 O Serotypes and Virulence Markers in High Fat Ground Beef (HFGB) Samples

Testing for the presence of E. coli O157:H7, STEC including Big 6 O serotypes, and virulence markers in HFGB was performed as follows. 10 ml of an enrichment broth per sample of HFGB was collected and nucleic acid was extracted using standard methods such as an AB PrepMan™ Ultra Sample Preparation Reagent according to the manufacturer's protocol. Each HFGB sample was assayed using real time PCR analysis as described herein employing the primers listed in Table 4 (below).

TABLE 4

| | | |
|---|---|---|
| E.coli 16S | TGG GAA CTG CAT CTG | (SEQ ID NO: 49) |
| rRNA-F | ATA CTG GCA | |
| E.coli 16S | TCT ACG CAT TTC ACC | (SEQ ID NO: 50) |
| rRNA-R | GCT ACA CCT | |
| Ecf1-F | CCC TTA TGA AGA GCC | (SEQ ID NO: 1) |
| | AGT ACT GAA G | |
| Ecf1-R | ATT ACG CAT AGG GCG | (SEQ ID NO: 2) |
| | TAT CAG CAC | |
| stx1F | ATA AAT CGC CAT TCG | (SEQ ID NO: 27) |
| | TTG ACT AC | |
| stx1R | AGA ACG CCC ACT GAG | (SEQ ID NO: 28) |
| | ATC ATC | |
| stx2F | GGC ACT GTC TGA AAC | (SEQ ID NO: 29) |
| | TGC TCC | |
| stx2R | TCG CCA GTT ATC TGA | (SEQ ID NO: 30) |
| | CAT TCT G | |
| eae-F | CAT TGA TCA GGA TTT | (SEQ ID NO: 31) |
| | TTC TGG TGA TA | |
| eae-R | CTC ATG CGG AAA TAG | (SEQ ID NO: 32) |
| | CCG TTA | |
| ehxA-F | GTG TCA GTA GGG AAG | (SEQ ID NO: 35) |
| | CGA ACA | |
| ehxA-R | ATC ATG TTT TCC GCC | (SEQ ID NO: 36) |
| | AAT G | |
| wzx-F | TGCGTGTGGCAAAAATTTA | (SEQ ID NO: 37) |
| | AAGAT | |
| wzx-R | GTTGCCAATCAATCATGCC | (SEQ ID NO: 38) |
| | AGAAG | |
| wzx158-O26-F | GTA TCG CTG AAA TTA | (SEQ ID NO: 51) |
| | GAA GCG C | |
| wzx158-O26-R | AGT TGA AAC ACC CGT | (SEQ ID NO: 52) |
| | AAT GGC | |
| wzx237-O111-F | TGT TCC AGG TGG TAG | (SEQ ID NO: 53) |
| | GAT TCG | |
| wzx237-O111-R | TCA CGA TGT TGA TCA | (SEQ ID NO: 54) |
| | TCT GGG | |
| wzx72-O45-F | CGT TGT GCA TGG TGG | (SEQ ID NO: 55) |
| | CAT | |
| wzx72-O45-R | TGG CCA AAC CAA CTA | (SEQ ID NO: 56) |
| | TGA ACT G | |
| wzx189-O121-F | AGG CGC TGT TTG GTC | (SEQ ID NO: 57) |
| | TCT TAG A | |
| wzx189-O121-R | GAA CCG AAA TGA TGG | (SEQ ID NO: 58) |
| | GTG CT | |
| wzx191-O103-F | TTG GAG CGT TAA CTG | (SEQ ID NO: 59) |
| | GAC CT | |
| wzx191-O103-R | ATA TTC GCT ATA TCT | (SEQ ID NO: 60) |
| | TCT TGC GGC | |
| wzx135-O145-F | AAA CTG GGA TTG GAC | (SEQ ID NO: 61) |
| | GTG G | |
| wzx135-O145-R | CCC AAA ACT TCT AGG | (SEQ ID NO: 62) |
| | CCC G | |

Results

The results are summarized in Table 5 (below).

TABLE 5
HFGB samples n = 644

| O157:H7/NM | Virulent STEC | Big 6 STEC |
|---|---|---|
| 0/644 | 26/644 | 25/644 |
| 0% positivity | 4% positivity | 3.9% positivity |

In 644 HFGB samples approximately 4.5% were stx, eae positive. This assay also detected several Big 6 STECs (O103, O26, O45, and O21). Furthermore, 23% of virulent non-O157:H7 STECs detected using this assay were non-Big 6 STECs. These included 5 stx/eae strains and an eae strain. This assay accordingly detects O157:H7, virulent O157:NM and virulent STECs including the Big 6 STECs. Here virulent STECs are defined genetically as those that are ecf positive which contain stx, eae, ehxA and a virulent plasmid.

### Example 3

### Colonization Factors

Additional testing using the methods described herein has revealed that ecf methodology detects enteropathogenic E. coli (EPEC strains such as E. coli with eae, ehxA, and ecf but not stx1 or stx2 or both shiga toxins) but not necessarily all, only those carrying the ecf operon genes. In view of such data, shiga toxins, eae and ecf are factors that enhance colonization of pathogenic E. coli in cattle. Such factors, however, are not mandatory so any one factor may be omitted (e.g., one or more shiga toxins in EPEC strains) and yet still colonize its host albeit at a lower frequency. Accordingly, although these genes (e.g., eae, ehxA, ecf, stx1, or stx2) are virulence factors in humans they are colonization factors in cattle.

### Example 4

### wzx_{O157} and ecf1

A combination of two unique target genes (wzx_{O157} and ecf1) has been identified as allowing for the specific detection of pathogenic E. coli O157:H7 strains. Here we described the sensitivity and specificity of an E. coli O157:H7 detection assay using a collection of 480 E. coli O157:H7 and non-pathogenic E. coli isolates of different serotypes.

Methods: The E. coli O157:H7 detection assay combines two unique target genes, the chromosomal wzx_{O157} gene and the ecf1 gene which is located in a conserved ecf operon on a large virulence plasmid. The large virulence plasmid is found in highly pathogenic EHEC strains. The ecf operon encodes 4 proteins involved in cell wall synthesis which enhances colonization of E. coli in cattle. The sensitivity of the assay was determined by using serial 10-fold dilutions of five different E. coli O157:H7 strains. The sensitivity or limit of detection (LOD) was defined using a 95% confidence interval. We also determined the specificity of the assay by testing 480 inclusive and exclusive E. coli isolates, consisting of 117 E. coli O157:H7 and O157:NM strains, 7 non-virulent E. coli O157:NM strains, and 356 pathogenic and non-pathogenic non-O157 E. coli isolates including 130 of the FSIS regulated big six STEC strains. All isolates were tested at a concentration of 1e8 CFU/ml. Serotypes and presence of virulence genes such as shiga toxins 1 and 2 (stx₁ and stx₂), intimin (eae) and enterohemolysin (ehxA) for all E. coli isolates included in this study were tested by PCR.

Results: The LOD of the E. coli O157:H7 detection assay was determined to be 1e3 CFU/mL. All 117 O157H7/NM strains containing stx genes and the eae gene were successfully detected by the assay. Seven O157:NM strains which lacked shiga toxin genes were not detected. Of the 356 non-O157:H7 E. coli isolates included in this study, none were detected by the E. coli O157:H7 detection assay.

Significance: The results of these studies show that the use of the ecf1 gene in conjunction with the wzx_{O157} gene accurately detects stx/eae containing pathogenic O157:H7/NM strains. These data demonstrate that the O157:H7 detection assay has 100% specificity and an analytical LOD of 1e3 CFU/mL.

### Other Embodiments

All publications and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth.

What is claimed is:

1. A method for assigning whether a sample includes Shiga-toxin producing E. coli (STEC), said method comprising the steps of:
a) providing nucleic acids from a sample;
b) detecting an O157-specific fragment and an ECF-specific fragment;
c) assigning to said sample one of the following outcomes:
i) if the O157-specific fragment and the ECF-specific fragment are absent then the sample is negative for virulent O157 STEC and a virulent non-O157:H7 STEC;
ii) if the O157-specific fragment is present and the ECF-specific fragment is absent then the sample is negative for a virulent non-O157:H7 STEC;
iii) if the O157-specific fragment and ECF-specific fragment are present then the sample includes virulent O157 STEC; or
iv) if the O157-specific fragment is absent and the ECF-specific fragment is present then the sample includes a virulent non-O157:H7 STEC.

2. The method of claim 1, wherein said O157-specific fragment is rfb, wzx, or wzy.

3. The method of claim 1, wherein said virulent O157 STEC includes O157:H7, O157:NM, O157:H-, O157:H8, or O157:H21.

4. The method of claim 1, wherein said virulent, non-O157:H7 STEC includes O26, O45, O103, O111, O121, or O145.

5. The method of claim 1, wherein said method involves detection of at least two O157-specific fragments (e.g., rfb and wzk, rfb and wzy, and wzk and wzy, or rfb, wzk, and wzy).

6. A method for assigning whether a sample includes STEC, said method comprising the steps of:
a) providing nucleic acids from a sample;
b) detecting an O157:H7-specific fragment and a ECF-specific fragment;
c) assigning to said sample one of the following outcomes:
i) if the O157:H7-specific fragment and the ECF-specific fragment are absent then the sample is negative for O157:H7 STEC and a virulent non-O157:H7 STEC is present;
ii) if the O157:H7-specific fragment is present and the ECF-specific fragment is absent then the sample is negative for a virulent non-O157:H7 STEC;
iii) if the O157:H7-specific fragment and the ECF-specific fragment are both present then the sample includes an O157:H7 STEC; or
iv) if the O157:H7-specific fragment is absent and the ECF-specific fragment is present then the sample includes a virulent non-O157:H7 STEC.

7. The method of claim 6, wherein said O157:H7-specific fragment includes katP junction or Z5866.

8. The method of claim 6, wherein said virulent, non-O157:H7 STEC includes O26, O45, O103, O111, O121, or O145.

9. The method of claim 6, wherein said method involves detection of at least two O157:H7-specific fragments.

10. A method of assigning whether a sample includes STEC, said method comprising the steps of:
a) providing nucleic acids from a sample;
b) detecting a first fragment that detects O157 STEC and STEC lacking an ECF gene, and a second fragment that detects an ECF gene;
c) assigning to said sample one of the following outcomes:
i) if the first and second fragments are absent then the sample is negative for virulent O157 STEC and a virulent non-O157:H7 STEC;
ii) if the first fragment is present and the second fragment is absent then the sample is negative for a virulent non-O157:H7 STEC;
iii) if the first fragment and second fragment are present then the sample includes virulent O157 STEC; or
iv) if the first fragment is absent and the second fragment is present then the sample includes a virulent non-O157:H7 STEC.

11. The method of claim 10, wherein said first fragment is Sil or Z0372.

12. The method of claim 10, wherein said virulent O157 STEC includes O157:H7, O157:NM, O157:H-, O157:H8, or O157:H21.

13. The method of claim 10, wherein said virulent, non-O157:H7 STEC includes O26, O45, O103, O111, O121, or O145.

14. The method of claim 10, wherein said method involves detection of at least two first fragments (e.g., Sil and Z0372).

15. A method of assigning whether a sample includes STEC, said method comprising the steps of:
a) obtaining nucleic acids from a sample;
b) detecting a first fragment that detects O157:H7 STEC and STEC lacking an ECF gene, and a second fragment that detects the ECF gene;
c) assigning to said sample one of the following outcomes:
i) if the first and second fragments are absent then the sample is negative for O157:H7 STEC and a virulent non-O157:H7 STEC;
ii) if the first fragment is present and the second fragment is absent then the sample is negative for virulent non-O157:H7 STEC;
iii) if the first fragment and second fragment are present then the sample includes an O157:H7 STEC; or
iv) if the first fragment is absent and the second fragment is present then the sample includes a virulent non-O157:H7 STEC.

16. The method of claim 15, wherein said virulent, non-O157:H7 STEC includes O26, O45, O103, O111, O121, or O145.

17. A method for detecting STEC in a sample, comprising the steps of:
a) providing a sample comprising nucleic acid molecules;
b) contacting said nucleic acid molecules with a virulent O157 STEC-specific probe and an ECF-specific probe under hybridization conditions, wherein
i) said virulent O157 STEC-specific probe specifically hybridizes to a virulent O157 STEC-specific fragment of said nucleic acid molecules; and
ii) said ECF-specific probe specifically hybridizes to an ECF-specific fragment of said nucleic acid molecules; and
c) detecting hybridization of said virulent O157 STEC-specific probe and said ECF-specific probe to identify the presence or absence of said virulent O157 STEC-specific fragment or said ECF-specific fragment as an indication of the presence of absence of STEC in the sample.

18. The method of claim 17, wherein the absence of said virulent O157 STEC-specific fragment and absence of said ECF-specific fragment is taken as an indication that the sample is negative for virulent O157 STEC and a virulent non-O157:H7 STEC.

19. The method of claim 17, wherein the presence of said virulent O157-specific fragment and the absence of said ECF-specific fragment is taken as an indication that the sample is negative for a virulent non-O157:H7 STEC.

20. The method of claim 17, wherein the presence of said virulent O157-specific fragment and the presence of said ECF-specific fragment is taken as an indication that the sample is positive for virulent O157 STEC.

21. The method of claim 17, wherein the absence of the virulent O157 STEC-specific fragment and the presence of the ECF-specific fragment is taken as an indication that the sample is positive for a virulent non-O157:H7 STEC.

22. The method of claim 17, wherein said virulent O157 STEC-specific fragment is rfb, wzx, or wzy.

23. The method of claim 17, wherein said virulent O157 STEC includes O157:H7, O157:NM, O157:H-, O157:H8, or O157:H21.

24. The method of claim 17, wherein said virulent, non-O157:H7 STEC includes O26, O45, O103, O111, O121, or O145.

25. The method of claim 17, wherein said method involves detection of at least two virulent O157 STEC-specific fragments (e.g., rfb and wzk, rfb and wzy, and wzk and wzy, or rfb, wzk, and wzy).

26. The method of claim 17, wherein said detecting hybridization involves amplification.

27. The method of claim 17, wherein said detecting hybridization involves cDNA synthesis.

28. The method of claim 17, wherein said nucleic acid molecules are purified from an environmental or a biological sample.

29. The method of claim 28, wherein said biological sample is a food sample.

30. The method of claim 29, wherein said food sample is a meat sample.

31. A method for detecting STEC in a sample, comprising the steps of:
a) providing a sample comprising nucleic acid molecules;
b) contacting said nucleic acid molecules with an O157:H7-specific probe and an ECF-specific probe under hybridization conditions, wherein
i) said O157:H7-specific probe specifically hybridizes to an O157:H7-specific fragment of said nucleic acid molecules; and
ii) said ECF-specific probe specifically hybridizes to an ECF-specific fragment of said nucleic acid molecules; and
c) detecting hybridization of said O157:H7-specific probe and said ECF-specific probe to identify the presence or absence of said O157:H7-specific fragment or said ECF-specific fragment as an indication of the presence of absence of STEC in the sample.

32. The method of claim 31, wherein the absence of said O157:H7-specific fragment and absence of said ECF-specific fragment is taken as an indication that the sample is negative for O157:H7 STEC and a virulent non-O157:H7 STEC.

33. The method of claim 31, wherein the presence of said O157:H7-specific fragment and the absence of said ECF-specific fragment is taken as an indication that the sample is negative for a virulent non-O157:H7 STEC.

34. The method of claim 31, wherein the presence of said O157:H7-specific fragment and the presence of said ECF-specific fragment is taken as an indication that the sample is positive for an O157:H7 STEC.

35. The method of claim 31, wherein the absence of the O157:H7-specific fragment and the absence of the ECF-specific fragment is taken as an indication that the sample is positive for a virulent non-O157:H7 STEC.

36. The method of claim 31, wherein said O157:H7-specific fragment includes katP junction or Z5866.

37. The method of claim 31, wherein said virulent, non-O157:H7 STEC includes O26, O45, O103, O111, O121, or O145.

38. The method of claim 31, wherein said method involves detection of at least two O157:H7-specific fragments (e.g, katP and Z5866).

39. The method of claim 31, wherein said detecting hybridization involves amplification.

40. The method of claim 31, wherein said detecting hybridization involves cDNA synthesis.

41. The method of claim 31, wherein said nucleic acid molecules are purified from an environmental or a biological sample.

42. The method of claim 41, wherein said biological sample is a food sample.

43. The method of claim 42, wherein said food sample is a meat sample.

44. A method for detecting STEC in a sample, comprising the steps of:
a) providing a sample comprising nucleic acid molecules;
b) contacting said nucleic acid molecules with a first probe and a second probe under hybridization conditions, wherein
i) said first probe specifically hybridizes with nucleic acid molecules of
(1) a virulent O157 STEC and
(2) STEC lacking an ECF gene; and
ii) said second probe specifically hybridizes to an ECF-specific fragment of said nucleic acid molecules; and
c) detecting hybridization of said first probe and said second probe, wherein the presence or absence of hybridization to said first probe and said second probe is taken as indication of the presence or absence of STEC in the sample.

45. The method of claim 44, wherein the absence of hybridization to said first probe and absence of hybridization to said second probe is taken as an indication that the sample is negative for virulent O157 STEC and a virulent non-O157:H7 STEC.

46. The method of claim 44, wherein the presence of hybridization to said first probe and the absence of hybridization to said second probe is taken as an indication that the sample is negative for a virulent non-O157:H7 STEC.

47. The method of claim 44, wherein the presence of hybridization to said first probe and the presence of hybridization to said second probe is taken as an indication that the sample is positive for virulent O157 STEC.

48. The method of claim 44, wherein the absence of hybridization to said first probe and the presence of hybridization to said second probe is taken as an indication that the sample is positive for a virulent non-O157:H7 STEC.

49. The method of claim 44, wherein said first fragment is Sil or Z0372.

50. The method of claim 44, wherein said virulent O157 STEC includes O157:H7, O157:NM, O157:H-, O157:H8, or O157:H21.

51. The method of claim 44, wherein said virulent, non-O157:H7 STEC includes O26, O45, O103, O111, O121, or O145.

52. The method of claim 44, wherein said method involves detection of at least two first fragments (e.g., Sil and Z0372).

53. The method of claim 44, wherein said detecting hybridization involves amplification.

54. The method of claim 44, wherein said detecting hybridization involves cDNA synthesis.

55. The method of claim 44, wherein said nucleic acid molecules are purified from an environmental or a biological sample.

56. The method of claim 55, wherein said biological sample is a food sample.

57. The method of claim 56, wherein said food sample is a meat sample.

58. A method for detecting STEC in a sample, comprising the steps of:
a) providing a sample comprising nucleic acid molecules;
b) contacting said nucleic acid molecules with a first probe and a second probe under hybridization conditions, wherein
i) said first probe specifically hybridizes with nucleic acid molecules of
(1) an O157:H7 STEC and
(2) STEC lacking an ECF gene; and
ii) said second probe specifically hybridizes to an ECF-specific fragment of said nucleic acid molecules; and
c) detecting hybridization of said first probe and said second probe, wherein the presence or absence of hybridization to said first probe and said second probe is taken as indication of the presence or absence of STEC in the sample.

59. The method of claim 8, wherein the absence of hybridization to said first probe and absence of hybridization to said second probe is taken as an indication that the sample is negative for O157 STEC and a virulent non-O157:H7 STEC.

60. The method of claim 58, wherein the presence of hybridization to said first probe and the absence of hybridization to said second probe is taken as an indication that the sample is negative for a virulent non-O157:H7 STEC.

61. The method of claim 58, wherein the presence of hybridization to said first probe and the presence of hybridization to said second probe is taken as an indication that the sample is positive for an O157:H7 STEC.

62. The method of claim 58, wherein the absence of hybridization to said first probe and the presence of hybridization to said second probe is taken as an indication that the sample is positive for a virulent non-O157:H7 STEC.

63. The method of claim 58, wherein said detecting hybridization involves amplification.

64. The method of claim 58, wherein said detecting hybridization involves cDNA synthesis.

65. The method of claim 58, wherein said nucleic acid molecules are purified from an environmental or a biological sample.

66. The method of claim 65, wherein said biological sample is a food sample.

67. The method of claim 66, wherein said food sample is a meat sample.

68. A method for assessing the presence or absence of virulent non-O157:H7 STEC in a sample, comprising the steps of:
a) contacting nucleic acid molecules from said sample with an ECF-specific probe under hybridization conditions, wherein said ECF-specific probe specifically hybridizes to an ECF-specific region; and
b) detecting hybridization of said ECF-specific probe and said nucleic acid molecules, wherein presence or absence of hybridization of said ECF-specific probe with said nucleic acid molecules indicates the presence or absence of virulent non-O157:H7 STEC in said sample.

69. The method of claim 68, wherein said nucleic acid molecules are contacted with a virulent O157 STEC-specific probe that specifically hybridizes to a virulent O157 STEC-specific fragment of said nucleic acid molecules, and wherein (i) absence of hybridization of said O157 STEC-specific probe and absence of hybridization of said ECF-specific probe is taken as an indication that the sample is negative for virulent O157 STEC and a virulent non-O157:H7 STEC; (ii) the presence of hybridization of said virulent O157-specific fragment and the absence of hybridization of said ECF-specific fragment is taken as an indication that the sample is negative for a virulent non-O157:H7 STEC; (iii) the presence of hybridization of said virulent O157-specific fragment and the presence of hybridization of said ECF-specific fragment is taken as an indication that the sample is positive for virulent O157 STEC; or (iv) the absence of hybridization of the virulent O157 STEC-specific fragment and the presence of hybridization of the ECF-specific fragment is taken as an indication that the sample is positive for a virulent non-O157:H7 STEC.

70. The method of claim 68, wherein said nucleic acid molecules are contacted with a O157:H7-specific probe that specifically hybridizes to an O157:H7-specific fragment of said nucleic acid molecules, and (i) the absence of hybridization of said O157:H7-specific fragment and absence of hybridization of said ECF-specific fragment is taken as an indication that the sample is negative for O157:H7 STEC and a virulent non-O157:H7 STEC; (ii) the presence of hybridization of said O157:H7-specific fragment and the absence of hybridization of said ECF-specific fragment is taken as an indication that the sample is negative for a virulent non-O157:H7 STEC; (iii) the presence of hybridization of said O157:H7-specific fragment and the presence of hybridization of said ECF-specific fragment is taken as an indication that the sample is positive for an O157:H7 STEC; and (iv) the absence of hybridization of the O157:H7-specific fragment and the absence of the ECF-specific fragment is taken as an indication that the sample is positive for a virulent non-O157:H7 STEC.

71. The method of claim 68, wherein said nucleic acid molecules are contacted with a probe (a′) that specifically hybridizes with nucleic acid molecules of (1) a virulent O157 STEC and (2) STEC lacking an ECF gene; and wherein (i) the absence of hybridization to said probe (a′) and absence of hybridization to said ECF-specific fragment is taken as an indication that the sample is negative for virulent O157 STEC and a virulent non-O157:H7 STEC, (ii) the presence of hybridization to said probe (a′) and the absence of hybridization to said ECF-specific fragment is taken as an indication that the sample is negative for a virulent non-O157:H7 STEC; (iii) the presence of hybridization to said probe (a′) and the presence of hybridization to said ECF-specific fragment is taken as an indication that the sample is positive for virulent O157 STEC, (iv) the absence of hybridization to said probe (a′) and the presence of hybridization to said ECF-specific fragment is taken as an indication that the sample is positive for a virulent non-O157:H7 STEC.

72. The method of claim 68, wherein said nucleic acid molecules are contacted with a probe (b′) that specifically hybridizes with nucleic acid molecules of (1) an O157:H7 STEC and (2) STEC lacking an ECF gene, and wherein (i) the absence of hybridization to probe (b′) and absence of hybridization to said ECF-specific fragment is taken as an indication that the sample is negative for O157 STEC and a virulent non-O157:H7 STEC; (ii) the presence of hybridization to said probe (b′) and the absence of hybridization to said ECF-specific fragment is taken as an indication that the sample is negative for a virulent non-O157:H7 STEC, (iii) the presence of hybridization to said probe (b′) and the presence of hybridization to said ECF-specific fragment is taken as an indication that the sample is positive for an O157:H7 STEC, and (iv) the absence of hybridization to said probe (b′) and the presence of hybridization to said ECF-specific fragment is taken as an indication that the sample is positive for a virulent non-O157:H7 STEC.

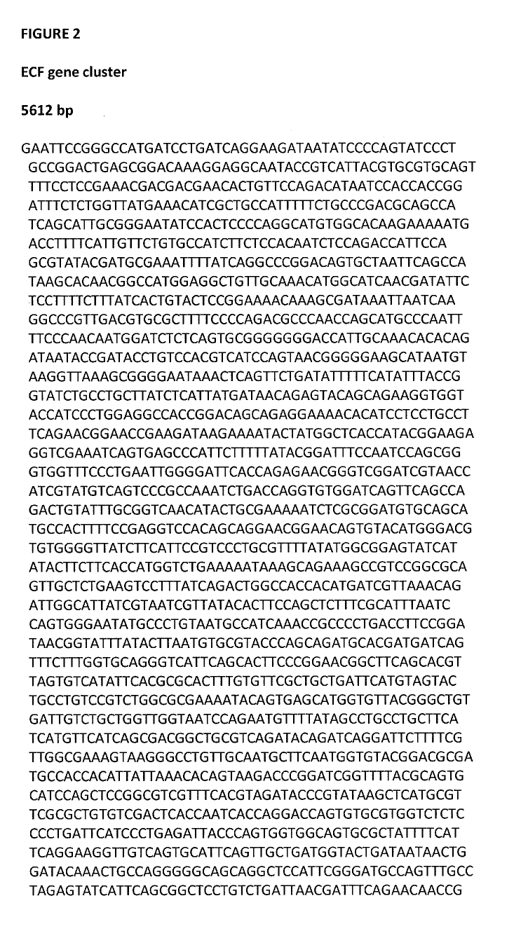

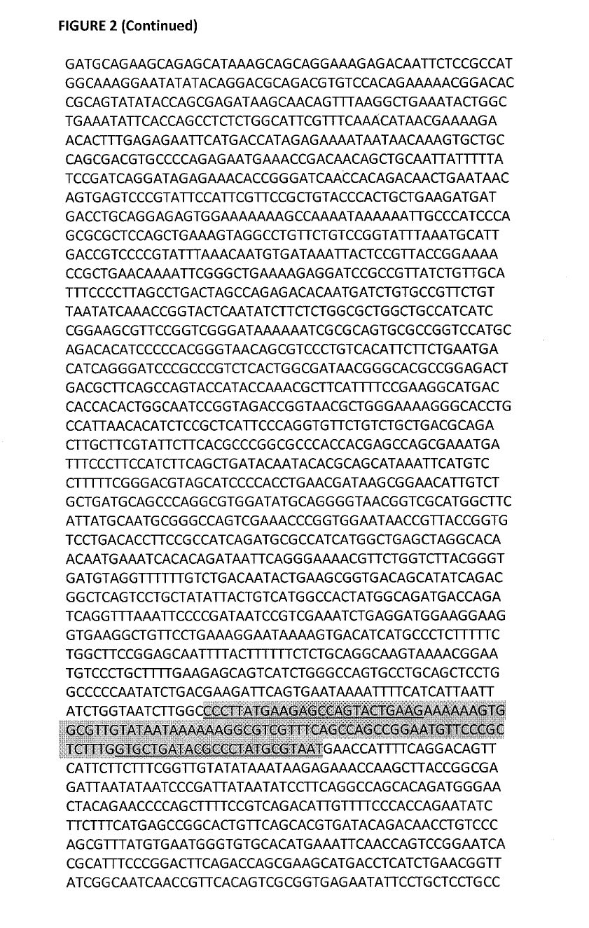

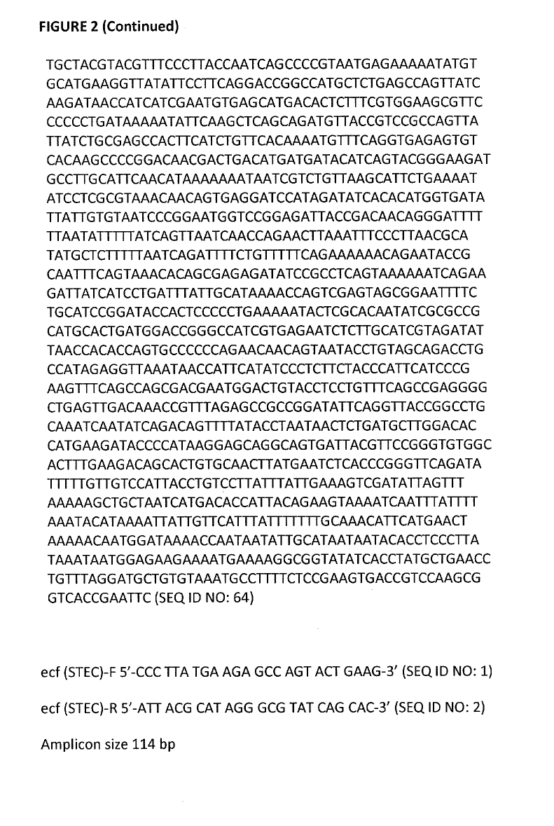

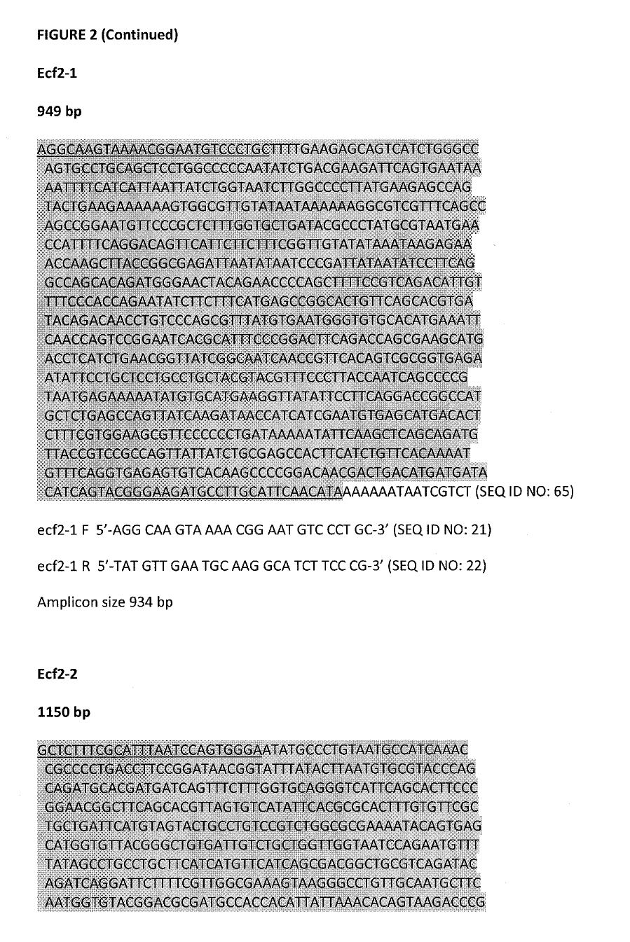

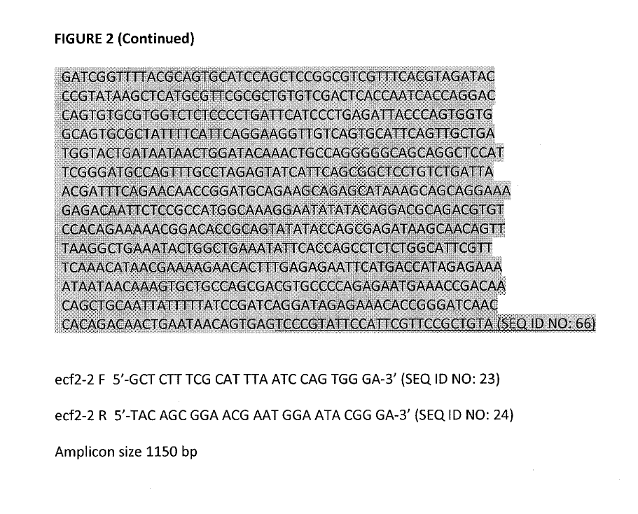

73. A composition comprising a nucleic acid consisting of a nucleic acid sequence wherein the nucleic acid sequence is a fragment of the Ecf gene cluster shown in FIG. 2 or a fragment thereof or sequence complementary thereto, wherein said fragment is 1-2404 bp or 3584-5612 bp as shown in FIG. 2.

74. The composition of claim 73, wherein the nucleic acid sequence is the 949 bp Ecf2-1 fragment.

75. The composition of claim 73, wherein the nucleic acid sequence is the 1050 bp Ecf2-2 fragment.

76. The composition of claim 73, wherein the nucleic acid is an isolated nucleic acid having a nucleotide sequence selected from the group consisting of:
(SEQ ID NO: 1)
5′-CCC TTA TGA AGA GCC AGT ACT GAA G-3′
and
(SEQ ID NO: 2)
5′ ATT ACG CAT AGG GCG TAT CAG CAC-3′.

77. A composition comprising a nucleic acid consisting of a nucleic acid sequence wherein the nucleic acid sequence is a 1318 bp Z5886 shown in FIG. 1 or a fragment thereof or sequence complementary thereto.

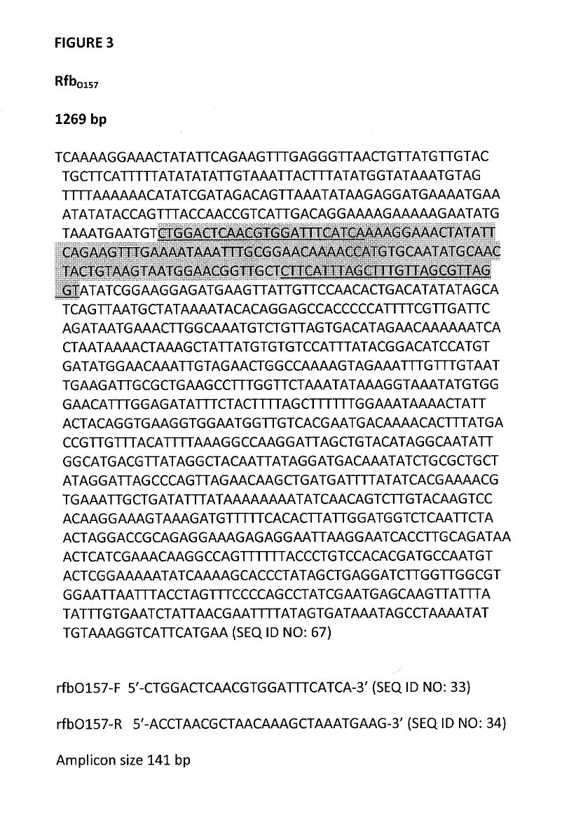

78. A composition comprising a nucleic acid consisting of a nucleic acid sequence wherein the nucleic acid sequence is a 1269 bp Rfb_{O157} shown in FIG. 3 or a fragment thereof or sequence complementary thereto.

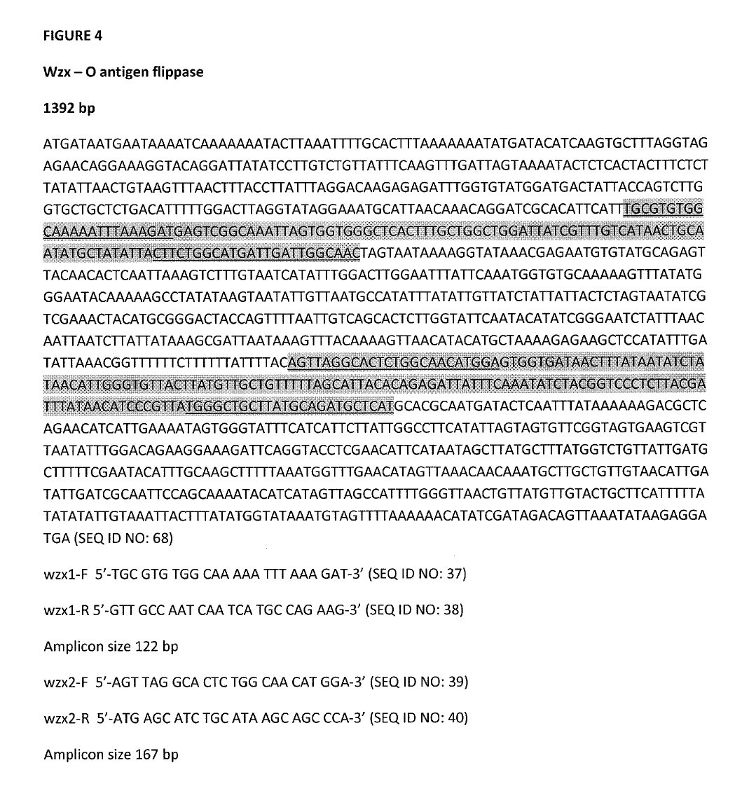

79. A composition comprising a nucleic acid consisting of a nucleic acid sequence wherein the nucleic acid sequence is a 1392 bp Wzx_{O157} shown in FIG. 4 or a fragment thereof or sequence complementary thereto.

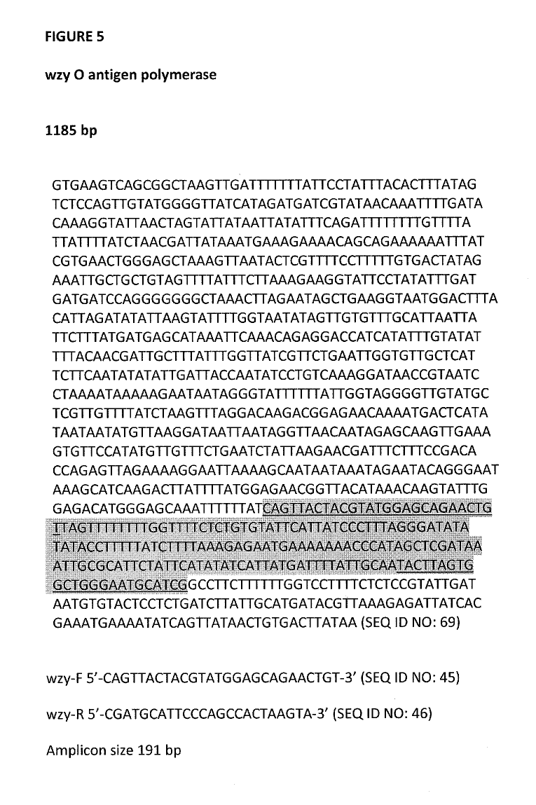

80. A composition comprising a nucleic acid consisting of a nucleic acid sequence wherein the nucleic acid sequence is a 1185 bp Wzy_{O157} shown in FIG. 5 or a fragment thereof or sequence complementary thereto.

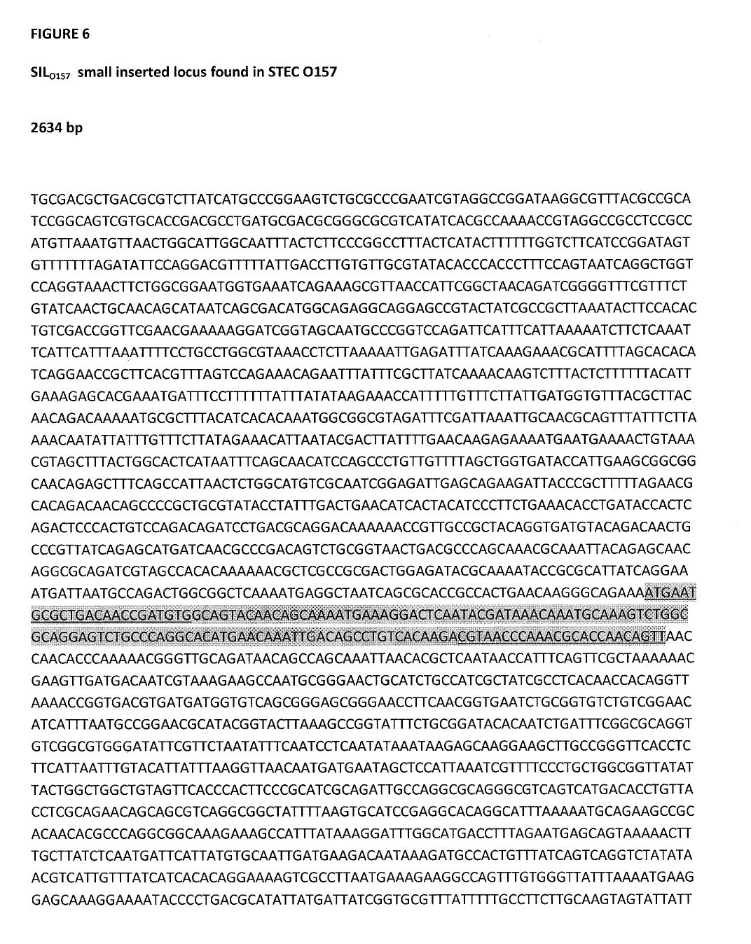

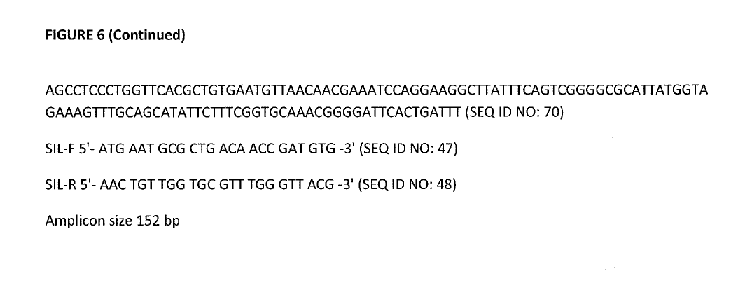

81. A composition comprising a nucleic acid consisting of a nucleic acid sequence wherein the nucleic acid sequence is a 2634 bp SIL_{O157} shown in FIG. 6 or a fragment thereof or sequence complementary thereto.

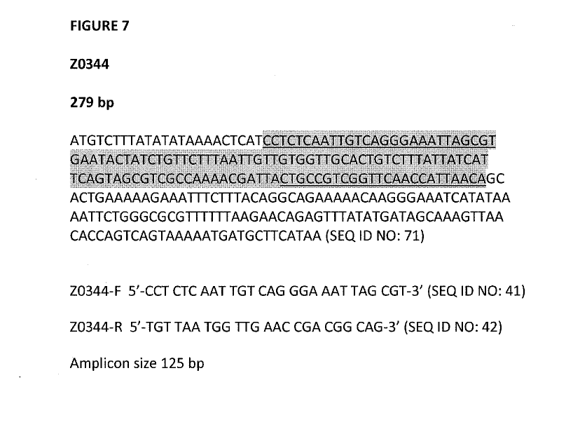

82. A composition comprising a nucleic acid consisting of a nucleic acid sequence wherein the nucleic acid sequence is a 279 bp Z0344 shown in FIG. 7 or a fragment thereof or sequence complementary thereto.

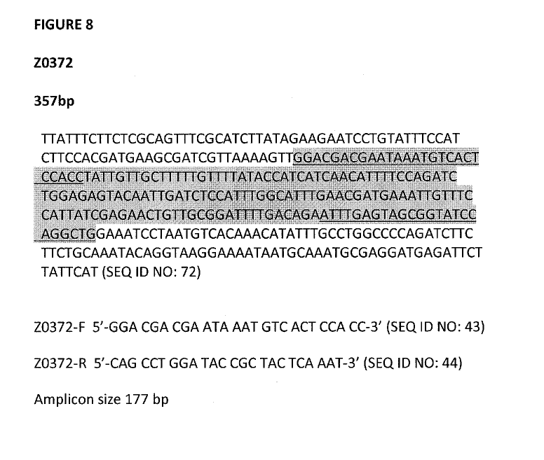

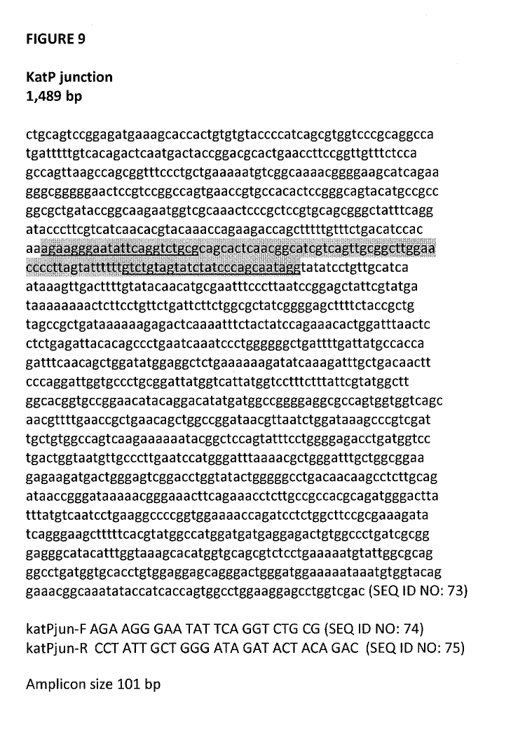

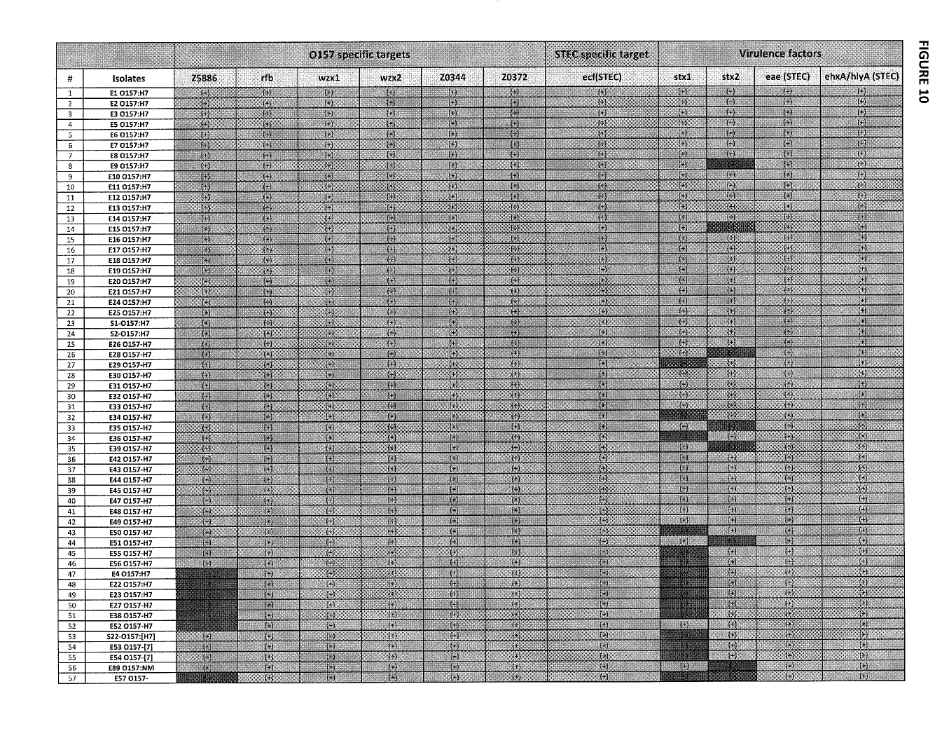

83. A composition comprising a nucleic acid consisting of a nucleic acid sequence wherein the nucleic acid sequence is a 357 bp Z0372 shown in FIG. 8 or a fragment thereof or sequence complementary thereto.

84. A composition, comprising: a first oligonucleotide that has a target-complementary base sequence to Ecf2-1 or Ecf2-2, optionally including a 5′ sequence that is not complementary to the specific target sequence.

85. A composition, comprising: a first oligonucleotide that has a target-complementary base sequence to Ecf gene cluster shown in FIG. 2, optionally including a 5′ sequence that is not complementary to the specific target sequence and a second oligonucleotide.

86. The composition of claim 85, wherein said second oligonucleotide is selected from the group consisting of:
a.) an oligonucleotide that has a target-complementary base sequence to Z5886, optionally including a 5′ sequence that is not complementary to the specific target sequence;
b.) an oligonucleotide that has a target-complementary base sequence to hylA, optionally including a 5′ sequence that is not complementary to the specific target sequence;
c.) an oligonucleotide that has a target-complementary base sequence to rfb_{O157}, optionally including a 5′ sequence that is not complementary to the specific target sequence;
d.) an oligonucleotide that has a target-complementary base sequence to wzx_{O157}, optionally including a 5′ sequence that is not complementary to the specific target sequence;
e.) an oligonucleotide that has a target-complementary base sequence to wzy_{O157}, optionally including a 5′ sequence that is not complementary to the specific target sequence;
f.) an oligonucleotide that has a target-complementary base sequence to SIL_{O157}, optionally including a 5′ sequence that is not complementary to the specific target sequence;
g.) an oligonucleotide that has a target-complementary base sequence to Z0344, optionally including a 5′ sequence that is not complementary to the specific target sequence;
h.) an oligonucleotide that has a target-complementary base sequence to Z0372, optionally including a 5′ sequence that is not complementary to the specific target sequence;
i.) an oligonucleotide that has a target-complementary base sequence to katP junction, optionally including a 5′ sequence that is not complementary to the specific target sequence.

87. The composition of claim 85, wherein only one of the first and second oligonucleotides has a 3′ end that can be extended by a template-dependent DNA polymerase.

88. The composition of claim 85, further comprising a detectably labeled hybridization probe.


SEQUENCE LISTING (75 sequences)

SEQ ID NO: 1 - DNA, 25 bp, Artificial Sequence
  Description of Artificial Sequence Synthetic primer
cccttatgaagagccagtactgaag

SEQ ID NO: 2 - DNA, 24 bp, Artificial Sequence
  Description of Artificial Sequence Synthetic primer
attacgcatagggcgtatcagcac

SEQ ID NO: 3 - DNA, 24 bp, Artificial Sequence
  Description of Artificial Sequence Synthetic primer
tgcaaggcatcttcccgtactgat

SEQ ID NO: 4 - DNA, 24 bp, Artificial Sequence
  Description of Artificial Sequence Synthetic primer
tctgcgagccacttcatctgttca

SEQ ID NO: 5 - DNA, 24 bp, Artificial Sequence
  Description of Artificial Sequence Synthetic primer
agcaggaatattctcaccgcgact

SEQ ID NO: 6 - DNA, 24 bp, Artificial Sequence
  Description of Artificial Sequence Synthetic primer
acagacaacctgtcccagcgttta

SEQ ID NO: 7 - DNA, 24 bp, Artificial Sequence
  Description of Artificial Sequence Synthetic primer
ttcctttgccatggcggagaattg

SEQ ID NO: 8 - DNA, 24 bp, Artificial Sequence
  Description of Artificial Sequence Synthetic primer
agcggctcctgtctgattaacgat

SEQ ID NO: 9 - DNA, 24 bp, Artificial Sequence
  Description of Artificial Sequence Synthetic primer
tgatcatcgtgcatctgctgggta

SEQ ID NO: 10 - DNA, 24 bp, Artificial Sequence
  Description of Artificial Sequence Synthetic primer
atgccctgtaatgccatcaaaccg

SEQ ID NO: 11 - DNA, 24 bp, Artificial Sequence
  Description of Artificial Sequence Synthetic primer
tgtacactgttccgttcctgctgt

SEQ ID NO: 12 - DNA, 24 bp, Artificial Sequence
  Description of Artificial Sequence Synthetic primer
tccctgaattgcggattcaccaga

SEQ ID NO: 13 - DNA, 24 bp, Artificial Sequence
  Description of Artificial Sequence Synthetic primer
acgctggaatggtctggagattgt

SEQ ID NO: 14 - DNA, 24 bp, Artificial Sequence
  Description of Artificial Sequence Synthetic primer
atccaccaccggatttctctggtt

SEQ ID NO: 15 - DNA, 24 bp, Artificial Sequence
  Description of Artificial Sequence Synthetic primer
aactttaccggttatcggacggct

SEQ ID NO: 16 - DNA, 24 bp, Artificial Sequence
  Description of Artificial Sequence Synthetic primer
tgctcaggatgtggacgaacgaaa

SEQ ID NO: 17 - DNA, 24 bp, Artificial Sequence
  Description of Artificial Sequence Synthetic primer
tggtaccaccttctgctgtactct

SEQ ID NO: 18 - DNA, 24 bp, Artificial Sequence
  Description of Artificial Sequence Synthetic primer
tacctgtccacgtcatccagtaac

SEQ ID NO: 19 - DNA, 25 bp, Artificial Sequence
  Description of Artificial Sequence Synthetic primer
ttaattttgatgccagccaggttgg

SEQ ID NO: 20 - DNA, 26 bp, Artificial Sequence
  Description of Artificial Sequence Synthetic primer
gctagattctgacgttattgctggtc

SEQ ID NO: 21 - DNA, 26 bp, Artificial Sequence
  Description of Artificial Sequence Synthetic primer
aggcaagtaaaacggaatgtccctgc

SEQ ID NO: 22 - DNA, 26 bp, Artificial Sequence
  Description of Artificial Sequence Synthetic primer
tatgttgaatgcaaggcatcttcccg

SEQ ID NO: 23 - DNA, 26 bp, Artificial Sequence
  Description of Artificial Sequence Synthetic primer
gctctttcgcatttaatccagtggga

SEQ ID NO: 24 - DNA, 26 bp, Artificial Sequence
  Description of Artificial Sequence Synthetic primer
tacagcggaacgaatggaatacggga

SEQ ID NO: 25 - DNA, 21 bp, Artificial Sequence
  Description of Artificial Sequence Synthetic primer
atctccaaggcggcaacgaaa

SEQ ID NO: 26 - DNA, 30 bp, Artificial Sequence
  Description of Artificial Sequence Synthetic primer
cagaaggttatgaagttgagttcattccag

SEQ ID NO: 27 - DNA, 23 bp, Artificial Sequence
  Description of Artificial Sequence Synthetic primer
ataaatcgccattcgttgactac

SEQ ID NO: 28 - DNA, 21 bp, Artificial Sequence
  Description of Artificial Sequence Synthetic primer
agaacgcccactgagatcatc

SEQ ID NO: 29 - DNA, 21 bp, Artificial Sequence
  Description of Artificial Sequence Synthetic primer
ggcactgtctgaaactgctcc

SEQ ID NO: 30 - DNA, 22 bp, Artificial Sequence
  Description of Artificial Sequence Synthetic primer
tcgccagttatctgacattctg

SEQ ID NO: 31 - DNA, 26 bp, Artificial Sequence
  Description of Artificial Sequence Synthetic primer
cattgatcaggatttttctggtgata

SEQ ID NO: 32 - DNA, 21 bp, Artificial Sequence
  Description of Artificial Sequence Synthetic primer
ctcatgcggaaatagccgtta

SEQ ID NO: 33 - DNA, 24 bp, Artificial Sequence
  Description of Artificial Sequence Synthetic primer
ctggactcaacgtggatttcatca

SEQ ID NO: 34 - DNA, 27 bp, Artificial Sequence
  Description of Artificial Sequence Synthetic primer
acctaacgctaacaaagctaaatgaag

SEQ ID NO: 35 - DNA, 21 bp, Artificial Sequence
  Description of Artificial Sequence Synthetic primer
gtgtcagtagggaagcgaaca

SEQ ID NO: 36 - DNA, 19 bp, Artificial Sequence
  Description of Artificial Sequence Synthetic primer
atcatgttttccgccaatg

SEQ ID NO: 37 - DNA, 24 bp, Artificial Sequence
  Description of Artificial Sequence Synthetic primer
tgcgtgtggcaaaaatttaaagat

SEQ ID NO: 38 - DNA, 24 bp, Artificial Sequence
  Description of Artificial Sequence Synthetic primer
gttgccaatcaatcatgccagaag

SEQ ID NO: 39 - DNA, 24 bp, Artificial Sequence
  Description of Artificial Sequence Synthetic primer
agttaggcactctggcaacatgga

SEQ ID NO: 40 - DNA, 24 bp, Artificial Sequence
  Description of Artificial Sequence Synthetic primer
atgagcatctgcataagcagccca

SEQ ID NO: 41 - DNA, 27 bp, Artificial Sequence
  Description of Artificial Sequence Synthetic primer
cctctcaattgtcagggaaattagcgt

SEQ ID NO: 42 - DNA, 24 bp, Artificial Sequence
  Description of Artificial Sequence Synthetic primer
tgttaatggttgaaccgacggcag

SEQ ID NO: 43 - DNA, 26 bp, Artificial Sequence
  Description of Artificial Sequence Synthetic primer
ggacgacgaataaatgtcactccacc

SEQ ID NO: 44 - DNA, 24 bp, Artificial Sequence
  Description of Artificial Sequence Synthetic primer
cagcctggataccgctactcaaat

SEQ ID NO: 45 - DNA, 27 bp, Artificial Sequence
  Description of Artificial Sequence Synthetic primer
cagttactacgtatggagcagaactgt

SEQ ID NO: 46 - DNA, 24 bp, Artificial Sequence
  Description of Artificial Sequence Synthetic primer
cgatgcattcccagccactaagta

SEQ ID NO: 47 - DNA, 24 bp, Artificial Sequence
  Description of Artificial Sequence Synthetic primer
atgaatgcgctgacaaccgatgtg

SEQ ID NO: 48 - DNA, 24 bp, Artificial Sequence
  Description of Artificial Sequence Synthetic primer
aactgttggtgcgtttgggttacg

SEQ ID NO: 49 - DNA, 24 bp, Artificial Sequence
  Description of Artificial Sequence Synthetic primer
tgggaactgcatctgatactggca

SEQ ID NO: 50 - DNA, 24 bp, Artificial Sequence
  Description of Artificial Sequence Synthetic primer
tctacgcatttcaccgctacacct

SEQ ID NO: 51 - DNA, 22 bp, Artificial Sequence
  Description of Artificial Sequence Synthetic primer
gtatcgctgaaattagaagcgc

SEQ ID NO: 52 - DNA, 21 bp, Artificial Sequence
  Description of Artificial Sequence Synthetic primer
agttgaaacacccgtaatggc

SEQ ID NO: 53 - DNA, 21 bp, Artificial Sequence
  Description of Artificial Sequence Synthetic primer
tgttccaggtggtaggattcg

SEQ ID NO: 54 - DNA, 21 bp, Artificial Sequence
  Description of Artificial Sequence Synthetic primer
tcacgatgttgatcatctggg

SEQ ID NO: 55 - DNA, 18 bp, Artificial Sequence
  Description of Artificial Sequence Synthetic primer
cgttgtgcatggtggcat

SEQ ID NO: 56 - DNA, 22 bp, Artificial Sequence
  Description of Artificial Sequence Synthetic primer
tggccaaaccaactatgaactg

SEQ ID NO: 57 - DNA, 22 bp, Artificial Sequence
  Description of Artificial Sequence Synthetic primer
aggcgctgtttggtctcttaga

SEQ ID NO: 58 - DNA, 20 bp, Artificial Sequence
  Description of Artificial Sequence Synthetic primer
gaaccgaaatgatgggtgct

SEQ ID NO: 59 - DNA, 20 bp, Artificial Sequence
  Description of Artificial Sequence Synthetic primer
ttggagcgttaactggacct

SEQ ID NO: 60 - DNA, 24 bp, Artificial Sequence
  Description of Artificial Sequence Synthetic primer
atattcgctatatcttcttgcggc

SEQ ID NO: 61 - DNA, 19 bp, Artificial Sequence
  Description of Artificial Sequence Synthetic primer
aaactgggattggacgtgg

SEQ ID NO: 62 - DNA, 19 bp, Artificial Sequence
  Description of Artificial Sequence Synthetic primer
cccaaaacttctaggcccg

SEQ ID NO: 63 - DNA, 1318 bp, Escherichia coli
ttaattttgatgccagccaggttggtcattctcaaatacctcagcctcggggaatattttagtcaatgggataacatatttgaccagatcatgggtaaaaaagtcatccgcagtttcatctaccatccacgcagagttctcatctatgcgtggcacgagataatgaacattcaggcggactattttagatttttgctcaaaatttactatggcagcatagtttttttcccttcgtttatcggcaacatccttttgaatatctacatccaaagctatatgccctacgcctagaccataatccaaaagctggttatctatctctgctaatgtttttgttacatgtcgcgaacgtgcatcgaaagatttttcattgatacaacgccatgttatcaaagaagcaagtttaactttggttatgaaacgtgagtcgcgctcatctggtcgtcccatagcaacaacttggtaattttcatcttccagaggggagcctttgataagacgagcaagtttagtgttgactagtaatgagccgttttcagtgatatcttccctgataagatttaagttggccggtctaatcgttccttttccataatcatcattccattcataaaatacacctttaaagttttttaagtggctaatgatatagttttcaggcgcgtctttaacttcacataaatatgtgacatcagtccatacgttcatctttttggatttgatatgaagttcggcaagatgtgagcgtatatgatgctgtaatatttcctgctttacgtaaggacctttctgaagccttttgcattcgacaaagaaataatcgccatctccaaggcggcaacgaaactccggggtttttgctatccctttttgttctggaatgaactcaacttcataaccttctgatgcatagtttccagcaagaaccaactctaataaggctgtgtctggtaatactgttgtattcttaagcattctgattgcgcgatctctagcgccagtaattctatcgagcgatttagcacacactcctaattgtttaacccatggtattatatttgaagcattagtaacttcgtaagaccttctattatcgataagcgattttgcttgtgcgaaataaccagcaaccacgtcggaaccataccattctgggtcaaactgtttcccgaaattggcagctttttgggttgtagctatgtaaaattgctgtgcttttgctaagcgctggtaaaagtgttgtttgttctcttgagttgatgctaaccactcaagaccagcaataacgtcagaatctagcataacaccgtcattcatt

SEQ ID NO: 64 - DNA, 5612 bp, Escherichia coli
gaattccgggccatgatcctgatcaggaagataatatccccagtatccctgccggactgagcggacaaaggaggcaataccgtcattacgtgcgtgcagttttcctccgaaacgacgacgaacactgttccagacataatccaccaccggatttctctggttatgaaacatcgctgccatttttctgcccgacgcagccatcagcattgcgggaatatccactccccaggcatgtggcacaagaaaaatgaccttttcattgttctgtgccatcttctccacaatctccagaccattccagcgtatacgatgcgaaattttatcaggcccggacagtgctaattcagccataagcacaacggccatggaggctgttgcaaacatggcatcaacgatattctccttttctttatcactgtactccggaaaacaaagcgataaattaatcaaggcccgttgacgtgcgcttttccccagacgcccaaccagcatgcccaattttcccaacaatggatctctcagtgcgggggggaccattgcaaacacacagataataccgatacctgtccacgtcatccagtaacgggggaagcataatgtaaggttaaagcggggaataaactcagttctgatatttttcatatttaccggtatctgcctgcttatctcattatgataacagagtacagcagaaggtggtaccatccctggaggccaccggacagcagaggaaaacacatcctcctgccttcagaacggaaccgaagataagaaaatactatggctcaccatacggaagaggtcgaaatcagtgagcccattctttttatacggatttccaatccagcgggtggtttccctgaattggggattcaccagagaacgggtcggatcgtaaccatcgtatgtcagtcccgccaaatctgaccaggtgtggatcagttcagccagactgtatttgcggtcaacatactgcgaaaaatctcgcggatgtgcagcatgccacttttccgaggtccacagcaggaacggaacagtgtacatgggacgtgtggggttatcttcattccgtccctgcgttttatatggcggagtatcatatacttcttcaccatggtctgaaaaataaagcagaaagccgtccggcgcagttgctctgaagtcctttatcagactggccaccacatgatcgttaaacagattggcattatcgtaatcgttatacacttccagctctttcgcatttaatccagtgggaatatgccctgtaatgccatcaaaccgcccctgaccttccggataacggtatttatacttaatgtgcgtacccagcagatgcacgatgatcagtttctttggtgcagggtcattcagcacttcccggaacggcttcagcacgttagtgtcatattcacgcgcactttgtgttcgctgctgattcatgtagtactgcctgtccgtctggcgcgaaaatacagtgagcatggtgttacgggctgtgattgtctgctggttggtaatccagaatgttttatagcctgcctgcttcatcatgttcatcagcgacggctgcgtcagatacagatcaggattcttttcgttggcgaaagtaagggcctgttgcaatgcttcaatggtgtacggacgcgatgccaccacattattaaacacagtaagacccggatcggttttacgcagtgcatccagctccggcgtcgtttcacgtagatacccgtataagctcatgcgttcgcgctgtgtcgactcaccaatcaccaggaccagtgtgcgtggtctctcccctgattcatccctgagattacccagtggtggcagtgcgctattttcattcaggaaggttgtcagtgcattcagttgctgatggtactgataataactggatacaaactgccagggggcagcaggctccattcgggatgccagtttgcctagagtatcattcagcggctcctgtctgattaacgatttcagaacaaccggatgcagaagcagagcataaagcagcaggaaagagacaattctccgccatggcaaaggaatatatacaggacgcagacgtgtccacagaaaaacggacaccgcagtatataccagcgagataagcaacagtttaaggctgaaatactggctgaaatattcaccagcctctctggcattcgtttcaaacataacgaaaagaacactttgagagaattcatgaccatagagaaaataataacaaagtgctgccagcgacgtgccccagagaatgaaaccgacaacagctgcaattatttttatccgatcaggatagagaaacaccgggatcaaccacagacaactgaataacagtgagtcccgtattccattcgttccgctgtacccactgctgaagatgatgacctgcaggagagtggaaaaaaagccaaaataaaaaattgcccatcccagcgcgctccagctgaaagtaggcctgttctgtccggtatttaaatgcattgaccgtccccgtatttaaacaatgtgataaattactccgttaccggaaaaccgctgaacaaaattcgggctgaaaagaggatccgccgttatctgttgcatttccccttagcctgactagccagagacacaatgatctgtgccgttctgttaatatcaaaccggtactcaatatcttctctggcgctggctgccatcatccggaagcgttccggtcgggataaaaaatcgcgcagtgcgccggtccatgcagacacatcccccacgggtaacagcgtccctgtcacattcttctgaatgacatcagggatcccgcccgtctcactggcgataacgggcacgccggagactgacgcttcagccagtaccataccaaacgcttcattttccgaaggcatgaccaccacactggcaatccggtagaccggtaacgctgggaaaagggcacctgccattaacacatctccgctcattcccaggtgttctgtctgctgacgcagacttgcttcgtattcttcacgcccggcgcccaccacgagccagcgaaatgatttcccttccatcttcagctgatacaatacacgcagcataaattcatgtcctttttcgggacgtagcatccccacctgaacgataagcggaacattgtctgctgatgcagcccaggcgtggatatgcaggggtaacggtcgcatggcttcattatgcaatgcgggccagtcgaaacccggtggaataaccgttaccggtgtcctgacaccttccgccatcagatgcgccatcatggctgagctaggcacaacaatgaaatcacacagataattcagggaaaacgttctggtcttacgggtgatgtaggttttttgtctgacaatactgaagcggtgacagcatatcagacggctcagtcctgctatattactgtcatggccactatggcagatgaccagatcaggtttaaattccccgataatccgtcgaaatctgaggatggaaggaaggtgaaggctgttcctgaaaggaataaaagtgacatcatgccctctttttctggcttccggagcaattttacttttttctctgcaggcaagtaaaacggaatgtccctgcttttgaagagcagtcatctgggccagtgcctgcagctcctggcccccaatatctgacgaagattcagtgaataaaattttcatcattaattatctggtaatcttggccccttatgaagagccagtactgaagaaaaaagtggcgttgtataataaaaaaggcgtcgtttcagccagccggaatgttcccgctctttggtgctgatacgccctatgcgtaatgaaccattttcaggacagttcattcttctttcggttgtatataaataagagaaaccaagcttaccggcgagattaatataatcccgattataatatccttcaggccagcacagatgggaactacagaaccccagcttttccgtcagacattgttttcccaccagaatatcttctttcatgagccggcactgttcagcacgtgatacagacaacctgtcccagcgtttatgtgaatgggtgtgcacatgaaattcaaccagtccggaatcacgcatttcccggacttcagaccagcgaagcatgacctcatctgaacggttatcggcaatcaaccgttcacagtcgcggtgagaatattcctgctcctgcctgctacgtacgtttcccttaccaatcagccccgtaatgagaaaaatatgtgcatgaaggttatattccttcaggaccggccatgctctgagccagttatcaagataaccatcatcgaatgtgagcatgacactctttcgtggaagcgttcccccctgataaaaatattcaagctcagcagatgttaccgtccgccagttattatctgcgagccacttcatctgttcacaaaatgtttcaggtgagagtgtcacaagccccggacaacgactgacatgatgatacatcagtacgggaagatgccttgcattcaacataaaaaaataatcgtctgttaagcattctgaaaatatcctcgcgtaaacaacagtgaggatccatagatatcacacatggtgatattattgtgtaatcccggaatggtccggagattaccgacaacagggattttttaatatttttatcagttaatcaaccagaacttaaatttcccttaacgcatatgctctttttaatcagattttctgtttttcagaaaaaacagaataccgcaatttcagtaaacacagcgagagatatccgcctcagtaaaaaatcagaagattatcatcctgatttattgcataaaaccagtcgagtagcggaattttctgcatccggataccactccccctgaaaaatactcgcacaatatcgcgccgcatgcactgatggaccgggccatcgtgagaatctcttgcatcgtagatattaaccacaccagtgccccccagaacaacagtaatacctgtagcagacctgccatagaggttaaataaccattcatatccctcttctacccattcatcccgaagtttcagccagcgacgaatggactgtacctcctgtttcagccgaggggctgagttgacaaaccgtttagagccgccggatattcaggttaccggcctgcaaatcaatatcagacagttttatacctaataactctgatgcttggacaccatgaagataccccataaggagcaggcagtgattacgttccgggtgtggcactttgaagacagcactgtgcaacttatgaatctcacccgggttcagatatttttgttgtccattacctgtccttatttattgaaagtcgatattagtttaaaaagctgctaatcatgacaccattacagaagtaaaatcaatttattttaaatacataaaattattgttcatttatttttttgcaaacattcatgaactaaaaacaatggataaaaccaataatattgcataataatacacctcccttataaataatggagaagaaaatgaaaaggcggtatatcacctatgctgaacctgtttaggatgctgtgtaaatgccttttctccgaagtgaccgtccaagcggtcaccgaattc

SEQ ID NO: 65 - DNA, 949 bp, Escherichia coli
aggcaagtaaaacggaatgtccctgcttttgaagagcagtcatctgggccagtgcctgcagctcctggcccccaatatctgacgaagattcagtgaataaaattttcatcattaattatctggtaatcttggccccttatgaagagccagtactgaagaaaaaagtggcgttgtataataaaaaaggcgtcgtttcagccagccggaatgttcccgctctttggtgctgatacgccctatgcgtaatgaaccattttcaggacagttcattcttctttcggttgtatataaataagagaaaccaagcttaccggcgagattaatataatcccgattataatatccttcaggccagcacagatgggaactacagaaccccagcttttccgtcagacattgttttcccaccagaatatcttctttcatgagccggcactgttcagcacgtgatacagacaacctgtcccagcgtttatgtgaatgggtgtgcacatgaaattcaaccagtccggaatcacgcatttcccggacttcagaccagcgaagcatgacctcatctgaacggttatcggcaatcaaccgttcacagtcgcggtgagaatattcctgctcctgcctgctacgtacgtttcccttaccaatcagccccgtaatgagaaaaatatgtgcatgaaggttatattccttcaggaccggccatgctctgagccagttatcaagataaccatcatcgaatgtgagcatgacactctttcgtggaagcgttcccccctgataaaaatattcaagctcagcagatgttaccgtccgccagttattatctgcgagccacttcatctgttcacaaaatgtttcaggtgagagtgtcacaagccccggacaacgactgacatgatgatacatcagtacgggaagatgccttgcattcaacataaaaaaataatcgtct

SEQ ID NO: 66 - DNA, 1150 bp, Escherichia coli
gctctttcgcatttaatccagtgggaatatgccctgtaatgccatcaaaccgcccctgaccttccggataacggtatttatacttaatgtgcgtacccagcagatgcacgatgatcagtttctttggtgcagggtcattcagcacttcccggaacggcttcagcacgttagtgtcatattcacgcgcactttgtgttcgctgctgattcatgtagtactgcctgtccgtctggcgcgaaaatacagtgagcatggtgttacgggctgtgattgtctgctggttggtaatccagaatgttttatagcctgcctgcttcatcatgttcatcagcgacggctgcgtcagatacagatcaggattcttttcgttggcgaaagtaagggcctgttgcaatgcttcaatggtgtacggacgcgatgccaccacattattaaacacagtaagacccggatcggttttacgcagtgcatccagctccggcgtcgtttcacgtagatacccgtataagctcatgcgttcgcgctgtgtcgactcaccaatcaccaggaccagtgtgcgtggtctctcccctgattcatccctgagattacccagtggtggcagtgcgctattttcattcaggaaggttgtcagtgcattcagttgctgatggtactgataataactggatacaaactgccagggggcagcaggctccattcgggatgccagtttgcctagagtatcattcagcggctcctgtctgattaacgatttcagaacaaccggatgcagaagcagagcataaagcagcaggaaagagacaattctccgccatggcaaaggaatatatacaggacgcagacgtgtccacagaaaaacggacaccgcagtatataccagcgagataagcaacagtttaaggctgaaatactggctgaaatattcaccagcctctctggcattcgtttcaaacataacgaaaagaacactttgagagaattcatgaccatagagaaaataataacaaagtgctgccagcgacgtgccccagagaatgaaaccgacaacagctgcaattatttttatccgatcaggatagagaaacaccgggatcaaccacagacaactgaataacagtgagtcccgtattccattcgttccgctgta

SEQ ID NO: 67 - DNA, 1269 bp, Escherichia coli
tcaaaaggaaactatattcagaagtttgagggttaactgttatgttgtactgcttcatttttatatatattgtaaattactttatatggtataaatgtagttttaaaaaacatatcgatagacagttaaatataagaggatgaaaatgaaatatataccagtttaccaaccgtcattgacaggaaaagaaaaagaatatgtaaatgaatgtctggactcaacgtggatttcatcaaaaggaaactatattcagaagtttgaaaataaatttgcggaacaaaaccatgtgcaatatgcaactactgtaagtaatggaacggttgctcttcatttagctttgttagcgttaggtatatcggaaggagatgaagttattgttccaacactgacatatatagcatcagttaatgctataaaatacacaggagccacccccattttcgttgattcagataatgaaacttggcaaatgtctgttagtgacatagaacaaaaaatcactaataaaactaaagctattatgtgtgtccatttatacggacatccatgtgatatggaacaaattgtagaactggccaaaagtagaaatttgtttgtaattgaagattgcgctgaagcctttggttctaaatataaaggtaaatatgtgggaacatttggagatatttctacttttagcttttttggaaataaaactattactacaggtgaaggtggaatggttgtcacgaatgacaaaacactttatgaccgttgtttacattttaaaggccaaggattagctgtacataggcaatattggcatgacgttataggctacaattataggatgacaaatatctgcgctgctataggattagcccagttagaacaagctgatgattttatatcacgaaaacgtgaaattgctgatatttataaaaaaaatatcaacagtcttgtacaagtccacaaggaaagtaaagatgtttttcacacttattggatggtctcaattctaactaggaccgcagaggaaagagaggaattaaggaatcaccttgcagataaactcatcgaaacaaggccagttttttaccctgtccacacgatgccaatgtactcggaaaaatatcaaaagcaccctatagctgaggatcttggttggcgtggaattaatttacctagtttccccagcctatcgaatgagcaagttatttatatttgtgaatctattaacgaattttatagtgataaatagcctaaaatattgtaaaggtcattcatgaa

SEQ ID NO: 68 - DNA, 1392 bp, Escherichia coli
atgataatgaataaaatcaaaaaaatacttaaattttgcactttaaaaaaatatgatacatcaagtgctttaggtagagaacaggaaaggtacaggattatatccttgtctgttatttcaagtttgattagtaaaatactctcactactttctcttatattaactgtaagtttaactttaccttatttaggacaagagagatttggtgtatggatgactattaccagtcttggtgctgctctgacatttttggacttaggtataggaaatgcattaacaaacaggatcgcacattcatttgcgtgtggcaaaaatttaaagatgagtcggcaaattagtggtgggctcactttgctggctggattatcgtttgtcataactgcaatatgctatattacttctggcatgattgattggcaactagtaataaaaggtataaacgagaatgtgtatgcagagttacaacactcaattaaagtctttgtaatcatatttggacttggaatttattcaaatggtgtgcaaaaagtttatatgggaatacaaaaagcctatataagtaatattgttaatgccatatttatattgttatctattattactctagtaatatcgtcgaaactacatgcgggactaccagttttaattgtcagcactcttggtattcaatacatatcgggaatctatttaacaattaatcttattataaagcgattaataaagtttacaaaagttaacatacatgctaaaagagaagctccatatttgatattaaacggttttttcttttttattttacagttaggcactctggcaacatggagtggtgataactttataatatctataacattgggtgttacttatgttgctgtttttagcattacacagagattatttcaaatatctacggtccctcttacgatttataacatcccgttatgggctgcttatgcagatgctcatgcacgcaatgatactcaatttataaaaaagacgctcagaacatcattgaaaatagtgggtatttcatcattcttattggccttcatattagtagtgttcggtagtgaagtcgttaatatttggacagaaggaaagattcaggtacctcgaacattcataatagcttatgctttatggtctgttattgatgctttttcgaatacatttgcaagctttttaaatggtttgaacatagttaaacaacaaatgcttgctgttgtaacattgatattgatcgcaattccagcaaaatacatcatagttagccattttgggttaactgttatgttgtactgcttcatttttatatatattgtaaattactttatatggtataaatgtagttttaaaaaacatatcgatagacagttaaatataagaggatga

SEQ ID NO: 69 - DNA, 1185 bp, Escherichia coli
gtgaagtcagcggctaagttgatttttttattcctatttacactttatagtctccagttgtatggggttatcatagatgatcgtataacaaattttgatacaaaggtattaactagtattataattatatttcagattttttttgttttattattttatctaacgattataaatgaaagaaaacagcagaaaaaatttatcgtgaactgggagctaaagttaatactcgttttcctttttgtgactatagaaattgctgctgtagttttatttcttaaagaaggtattcctatatttgatgatgatccagggggggctaaacttagaatagctgaaggtaatggactttacattagatatattaagtattttggtaatatagttgtgtttgcattaattattctttatgatgagcataaattcaaacagaggaccatcatatttgtatattttacaacgattgctttatttggttatcgttctgaattggtgttgctcattcttcaatatatattgattaccaatatcctgtcaaaggataaccgtaatcctaaaataaaaagaataatagggtattttttattggtaggggttgtatgctcgttgttttatctaagtttaggacaagacggagaacaaaatgactcatataataatatgttaaggataattaataggttaacaatagagcaagttgaaagtgttccatatgttgtttctgaatctattaagaacgatttctttccgacaccagagttagaaaaggaattaaaagcaataataaatagaatacagggaataaagcatcaagacttattttatggagaacggttacataaacaagtatttggagacatgggagcaaattttttatcagttactacgtatggagcagaactgttagttttttttggttttctctgtgtattcattatccctttagggatatatatacctttttatcttttaaagagaatgaaaaaaacccatagctcgataaattgcgcattctattcatatatcattatgattttattgcaatacttagtggctgggaatgcatcggccttcttttttggtccttttctctccgtattgataatgtgtactcctctgatcttattgcatgatacgttaaagagattatcacgaaatgaaaatatcagttataactgtgacttataa

SEQ ID NO: 70 - DNA, 2634 bp, Escherichia coli
tgcgacgctgacgcgtcttatcatgcccggaagtctgcgcccgaatcgtaggccggataaggcgtttacgccgcatccggcagtcgtgcaccgacgcctgatgcgacgcgggcgcgtcatatcacgccaaaaccgtaggccgcctccgccatgttaaatgttaactggcattggcaatttactcttcccggcctttactcatacttttttggtcttcatccggatagtgtttttttagatattccaggacgtttttattgaccttgtgttgcgtatacacccaccctttccagtaatcaggctggtccaggtaaacttctggcggaatggtgaaatcagaaagcgttaaccattcggctaacagatcggggtttcgtttctgtatcaactgcaacagcataatcagcgacatggcagaggcaggagccgtactatcgccgcttaaatacttccacactgtcgaccggttcgaacgaaaaaggatcggtagcaatgcccggtccagattcatttcattaaaaatcttctcaaattcattcatttaaattttcctgcctggcgtaaacctcttaaaaattgagatttatcaaagaaacgcattttagcacacatcaggaaccgcttcacgtttagtccagaaacagaatttatttcgcttatcaaaacaagtctttactcttttttacattgaaagagcacgaaatgatttccttttttatttatataagaaaccatttttgtttcttattgatggtgtttacgcttacaacagacaaaaatgcgctttacatcacacaaatggcggcgtagatttcgattaaattgcaacgcagtttatttcttaaaacaatattatttgtttcttatagaaacattaatacgacttattttgaacaagagaaaatgaatgaaaactgtaaacgtagctttactggcactcataatttcagcaacatccagccctgttgttttagctggtgataccattgaagcggcggcaacagagctttcagccattaactctggcatgtcgcaatcggagattgagcagaagattacccgctttttagaacgcacagacaacagccccgctgcgtatacctatttgactgaacatcactacatcccttctgaaacacctgataccactcagactcccactgtccagacagatcctgacgcaggacaaaaaaccgttgccgctacaggtgatgtacagacaactgcccgttatcagagcatgatcaacgcccgacagtctgcggtaactgacgcccagcaaacgcaaattacagagcaacaggcgcagatcgtagccacacaaaaaacgctcgccgcgactggagatacgcaaaataccgcgcattatcaggaaatgattaatgccagactggcggctcaaaatgaggctaatcagcgcaccgccactgaacaagggcagaaaatgaatgcgctgacaaccgatgtggcagtacaacagcaaaatgaaaggactcaatacgataaacaaatgcaaagtctggcgcaggagtctgcccaggcacatgaacaaattgacagcctgtcacaagacgtaacccaaacgcaccaacagttaaccaacacccaaaaacgggttgcagataacagccagcaaattaacacgctcaataaccatttcagttcgctaaaaaacgaagttgatgacaatcgtaaagaagccaatgcgggaactgcatctgccatcgctatcgcctcacaaccacaggttaaaaccggtgacgtgatgatggtgtcagcgggagcgggaaccttcaacggtgaatctgcggtgtctgtcggaacatcatttaatgccggaacgcatacggtacttaaagccggtatttctgcggatacacaatctgatttcggcgcaggtgtcggcgtgggatattcgttctaatatttcaatcctcaatataaataagagcaaggaagcttgccgggttcacctcttcattaatttgtacattatttaaggttaacaatgatgaatagctccattaaatcgttttccctgctggcggttatattactggctggctgtagttcacccacttcccgcatcgcagattgccaggcgcagggcgtcagtcatgacacctgttacctcgcagaacagcagcgtcaggcggctattttaagtgcatccgaggcacaggcatttaaaaatgcagaagccgcacaacacgcccaggcggcaaagaaagccatttataaaggatttggcatgacctttagaatgagcagtaaaaactttgcttatctcaatgattcattatgtgcaattgatgaagacaataaagatgccactgtttatcagtcaggtctatataacgtcattgtttatcatcacacaggaaaagtcgccttaatgaaagaaggccagtttgtgggttatttaaaatgaaggagcaaaggaaaatacccctgacgcatattatgattatcggtgcgtttatttttgccttcttgcaagtagtattattagcctccctggttcacgctgtgaatgttaacaacgaaatccaggaaggcttatttcagtcggggcgcattatggtagaaagtttgcagcatattctttcggtgcaaacggggattcactgattt

SEQ ID NO: 71 - DNA, 279 bp, Escherichia coli
atgtctttatatataaaactcatcctctcaattgtcagggaaattagcgtgaatactatctgttctttaattgttgtggttgcactgtctttattatcattcagtagcgtcgccaaaacgattactgccgtcggttcaaccattaacagcactgaaaaagaaatttctttacaggcagaaaaacaagggaaatcatataaaattctgggcgcgttttttaagaacagagtttatatgatagcaaagttaacaccagtcagtaaaaatgatgcttcataa

SEQ ID NO: 72 - DNA, 357 bp, Escherichia coli
ttatttcttctcgcagtttcgcatcttatagaagaatcctgtatttccatcttccacgatgaagcgatcgttaaaagttggacgacgaataaatgtcactccacctattgttgctttttgttttataccatcatcaacattttccagatctggagagtacaattgatctccatttggcatttgaacgatgaaattgtttccattatcgagaactgttgcggattttgacagaatttgagtagcggtatccaggctggaaatcctaatgtcacaaacatatttgcctggccccagatcttcttctgcaaatacaggtaaggaaaataatgcaaatgcgaggatgagattcttattcat

SEQ ID NO: 73 - DNA, 1489 bp, Escherichia coli
ctgcagtccggagatgaaagcaccactgtgtgtaccccatcagcgtggtcccgcaggccatgatttttgtcacagactcaatgactaccggacgcactgaaccttccggttgtttctccagccagttaagccagcggtttccctgctgaaaaatgtcggcaaaacggggaagcatcagaagggcgggggaactccgtccggccagtgaaccgtgccacactccgggcagtacatgccgccggcgctgataccggcaagaatggtcgcaaactcccgctccgtgcagcgggctatttcaggatacccttcgtcatcaacacgtacaaaccagaagaccagctttttgtttctgacatccacaaagaagggaatattcaggtctgcgcagcactcaacggcatcgtcagttgcggcttggaaccccttagtattttttgtctgtagtatctatcccagcaataggtatatcctgttgcatcaataaagttgacttttgtatacaacatgcgaatttcccttaatccggagctattcgtatgataaaaaaaactcttcctgttctgattcttctggcgctatcggggagcttttctaccgctgtagccgctgataaaaaagagactcaaaatttctactatccagaaacactggatttaactcctctgagattacacagccctgaatcaaatccctggggggctgattttgattatgccaccagatttcaacagctggatatggaggctctgaaaaaagatatcaaagatttgctgacaacttcccaggattggtgccctgcggattatggtcattatggtcctttctttattcgtatggcttggcacggtgccggaacatacaggacatatgatggccggggaggcgccagtggtggtcagcaacgttttgaaccgctgaacagctggccggataacgttaatctggataaagcccgtcgattgctgtggccagtcaagaaaaaatacggctccagtatttcctggggagacctgatggtcctgactggtaatgttgcccttgaatccatgggatttaaaacgctgggatttgctggcggaagagaagatgactgggagtcggacctggtatactgggggcctgacaacaagcctcttgcagataaccgggataaaaacgggaaacttcagaaacctcttgccgccacgcagatgggacttatttatgtcaatcctgaaggccccggtggaaaaccagatcctctggcttccgcgaaagatatcagggaagctttttcacgtatggccatggatgatgaggagactgtggccctgatcgcgggagggcatacatttggtaaagcacatggtgcagcgtctcctgaaaaatgtattggcgcagggcctgatggtgcacctgtggaggagcagggactgggatggaaaaataaatgtggtacaggaaacggcaaatataccatcaccagtggcctggaaggagcctggtcgac

SEQ ID NO: 74 - DNA, 23 bp, Artificial Sequence
  Description of Artificial Sequence Synthetic primer
agaagggaatattcaggtctgcg

SEQ ID NO: 75 - DNA, 27 bp, Artificial Sequence
  Description of Artificial Sequence Synthetic primer
cctattgctgggatagatactacagac